(19) United States
(12) Patent Application Publication
(10) Pub. No.: US 2023/0172470 A1
(43) Pub. Date: Jun. 8, 2023

(54) APPARATUS AND METHOD FOR MEASURING MUSCLE OXYGEN CONSUMPTION

(71) Applicant: Panasonic Intellectual Property Management Co., Ltd., Osaka (JP)

(72) Inventor: HIROTO YANAGAWA, Osaka (JP)

(21) Appl. No.: 18/163,276

(22) Filed: Feb. 1, 2023

Related U.S. Application Data
(63) Continuation of application No. PCT/JP2021/028692, filed on Aug. 3, 2021.

(30) Foreign Application Priority Data
Aug. 26, 2020 (JP) 2020-142516

(51) Int. Cl. A61B 5/026; G16H 20/30; G16H 10/60; A61B 90/00

(52) U.S. Cl. CPC A61B 5/0261; A61B 90/36; G16H 10/60; G16H 20/30; A61B 2090/365

(57) ABSTRACT

A measurement apparatus includes a light source, a sensor with photoelectric converters, and a processing circuit. The processing circuit executes a detection operation multiple times to acquire multiple detection signals. The detection operation includes: causing the light source to emit a light pulse; and causing the sensor to detect at least part of an internal scattering component of a reflected light pulse, and output a detection signal representing a spatial distribution of intensity of the at least part of the internal scattering component. The reflected light pulse arises from the target portion due to emission of the light pulse. The internal scattering component is a component scattered in an interior of the target portion. The detection signal is included in the multiple detection signals. The processing circuit, based on the multiple detection signals, generates and outputs distribution data representing a spatial distribution of muscle oxygen consumption in the target portion.

### BACKGROUND

### 1. Technical Field

The present disclosure relates to an apparatus and a method for measuring muscle oxygen consumption.

### 2. Description of the Related Art

Recent years have seen increased demand for health clubs around the world, including Japan. According to a report by the International Health, Racquet and Sportsclub Association, the top country in terms of the number of health club members is the United States with approximately 54 million members (approximately 17.4% of the population). The ranking is followed by Germany, United Kingdom, and Brazil, with Japan having 4.16 million members (3.3% of the population). An increasing demand also exists for rehabilitation aimed at training the elderly to restore their functionality or helping the elderly to realize their potential. For such strength training and rehabilitation, improved training effectiveness is expected to be achieved through visualization of training effectiveness and through feedback to the training menu.

Vertebrates including human beings have blood circulatory systems. Such a blood circulatory system is known to dramatically increase in-vivo oxygen-carrying capacity to support the oxygen demands of cells including muscles. Invasive methods have traditionally been used for measurement of oxygen in the blood in a living body (see, for example, Gayeski, T. E. J., Honig C. R. “Direct measurements of intracellular O₂ gradients; role of convection and myoglobin”, 1983, Adv Med Biol.). Invasive methods allow for direct measurement of intravascular and intracellular oxygen dynamics through, for example, insertion of a catheter or an oxygen needle electrode. Unfortunately, the invasive nature of such a method makes it difficult to apply the method to strength training or rehabilitation.

To address this, attempts have been made in recent years to use near-infrared spectroscopy (NIRS) for non-invasive measurement of oxygen dynamics. Near-infrared light easily passes through a living body, and its absorption characteristics vary with the oxygenated or deoxygenated state of hemoglobin. Exploiting this property of near-infrared light makes it possible to keep track of in-vivo oxygen dynamics. For example, International Publication No. 2020/004020 and Japanese Unexamined Patent Application Publication No. 6-142087 each disclose an apparatus that employs NIRS to keep track of the state of intramuscular blood flow. International Publication No. 2020/004020 discloses a system that analyzes a change of intramuscular blood flow associated with muscle load exercise performed by a subject. Japanese Unexamined Patent Application Publication No. 6-142087 discloses an exercise monitor that detects muscular fatigue and provides an indication of an optimal exercise load.

### SUMMARY

One non-limiting and exemplary embodiment provides a technique for acquiring information on a two-dimensional distribution of muscle oxygen consumption by means of a simple method.

In one general aspect, the techniques disclosed here feature a measurement apparatus for measuring muscle oxygen consumption in a target portion of a user who performs a muscle exercise. The measurement apparatus includes a light source, a sensor, and a processing circuit. The sensor includes photoelectric converters. The processing circuit executes a detection operation multiple times to acquire multiple detection signals. The detection operation includes: causing the light source to emit a light pulse; and causing the sensor to detect at least part of an internal scattering component of a reflected light pulse, and to output a detection signal representing a spatial distribution of intensity of the at least part of the internal scattering component, the reflected light pulse arising from the target portion due to emission of the light pulse, the internal scattering component being a component scattered in an interior of the target portion, the detection signal being included in the multiple detection signals. The processing circuit, based on the multiple detection signals, generates and outputs distribution data representing a spatial distribution of muscle oxygen consumption in the target portion.

An aspect of the present disclosure makes it possible to acquire information on a two-dimensional distribution of muscle oxygen consumption by means of a simple method.

It should be noted that general or specific aspects of the present disclosure may be implemented as a system, an apparatus, a device, a method, an integrated circuit, a computer program, a recording medium such as a computer-readable recording disk, or any selective combination thereof. Examples of computer-readable recording media may include non-volatile recording media such as a Compact Disc-Read Only Memory (CD-ROM). The apparatus or device may be made up of one or more apparatuses or devices. If the apparatus or device is made up of two or more apparatuses or devices, the two or more apparatuses or devices may be disposed in a single piece of equipment, or may be disposed separately in two or more discrete pieces of equipment. As used herein and in the claims, the term “apparatus” or “device” may mean not only a single apparatus or device but also a system including multiple apparatuses or devices.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 illustrates the configuration of a measurement system according to an exemplary embodiment;

FIG. 2 illustrates an example of the time variation of the intensity of light arriving at an image sensor;

FIG. 3 illustrates an exemplary relationship between the duration of an input light pulse, and the quantity of light detected by the image sensor;

FIG. 4 illustrates an exemplary schematic configuration of one pixel of the image sensor;

FIG. 5 illustrates an exemplary configuration of the image sensor;

FIG. 6A illustrates an exemplary detection operation performed by using two wavelengths of light;

FIG. 6B illustrates another exemplary detection operation performed by using two wavelengths of light;

FIG. 7 illustrates waveforms of the optical intensity of a reflected light pulse;

FIG. 8A is a timing chart illustrating an exemplary operation of detecting an internal scattering component;

FIG. 8B is a timing chart illustrating an exemplary operation of detecting a surface reflection component;

FIG. 9 is a flowchart illustrating an exemplary operation performed by a control circuit operates to control a light source and the image sensor;

FIG. 10 schematically illustrates an example of the time variation of muscle blood flow;

FIG. 11 schematically illustrates an exemplary case where measurements are made simultaneously at multiple locations within a target portion;

FIG. 12 schematically illustrates an exemplary region to be irradiated with light;

FIG. 13 schematically illustrates how a signal changes in response to a lateral shift in the location of the target portion;

FIG. 14 illustrates how a single measurement operation of muscle oxygen consumption proceeds;

FIG. 15 is a flowchart illustrating an exemplary operation performed by the measurement apparatus to determine training effectiveness;

FIG. 16 illustrates an experiment for evaluating training effectiveness;

FIG. 17 illustrates an experiment protocol;

FIG. 18 illustrates an exemplary infrared image captured with a NIRS camera;

FIG. 19 illustrates the time variation of changes in hemoglobin level measured before training;

FIG. 20 illustrates the time variation of changes in hemoglobin level measured after training;

FIG. 21 illustrates the results of analysis on pre-training Oxy-Hb behavior for a case where the analysis period is 60 seconds;

FIG. 22 illustrates the results of analysis on post-training Oxy-Hb behavior for a case where the analysis period is 60 seconds;

FIG. 23 illustrates the results of analysis on pre-training Oxy-Hb behavior for a case where the analysis period is 30 seconds; and

FIG. 24 illustrates the results of analysis on post-training Oxy-Hb behavior for a case where the analysis period is 30 seconds.

### DETAILED DESCRIPTIONS

Embodiments described below each represent a generic or specific example. Specific details set forth in the following description of embodiments, such as numeric values, shapes, materials, components, the positioning and connection of components, steps, and the order of steps, are for illustrative purposes only and not intended to limit the technique according to the present disclosure. Those components in the following description of embodiments which are not cited in the independent claim representing the most generic concept of the present disclosure will be described as optional components. The figures are schematic and not necessarily to exact scale. Further, in the figures, the same reference signs are used to designate substantially the same or similar components. Repetitive descriptions will be omitted or simplified in some cases.

According to the present disclosure, each circuit, unit, apparatus, device, component, or part, or each functional unit in block diagrams may, in whole or in part, be implemented by one or more electronic circuits including, for example, a semiconductor device, a semiconductor integrated circuit (IC), or a large scale integration (LSI). An LSI or an IC may be integrated in a single chip or may be a combination of multiple chips. For example, functional blocks other than a memory element may be integrated in a single chip. Although herein called LSI or IC, such electronic circuit is called differently depending on the degree of integration, and may be an electronic circuit called a system LSI, a very large scale integration (VLSI), or ultra large scale integration (ULSI). A field programmable gate array (FPGA), which is programmed after manufacture of an LSI, or a reconfigurable logic device, which allows reconfiguration of connections inside an LSI or allows set-up of circuit segments inside an LSI, may be used for the same purpose.

Further, the functions or operations of circuits, units, apparatuses, devices, components, or parts may, in whole or in part, be implemented by software processing. In this case, the software is stored in one or more non-transitory recording media such as ROMs, optical disks, or hard disk drives, and when the software is executed by a processor, functions specified in the software are executed by the processor and peripheral devices. A system, or apparatus or device may include one or more non-transitory recording media in which the software is stored, a processor, and a required hardware device, such as an interface.

### Underlying Knowledge Forming Basis of the Present Disclosure

Prior to the description of embodiments of the present disclosure, the underlying knowledge forming the basis of the present disclosure will be described first.

Recently, it has been contemplated to evaluate the effectiveness of muscle exercise by measuring blood flow dynamics in human skeletal muscles at rest and during exercise by means of near-infrared spectroscopy (NIRS). Measurement of blood flow dynamics by NIRS is based on the high tissue penetration of near-infrared light and the wavelength dependence of the absorption coefficient of hemoglobin.

Near-infrared light exhibits relatively low coefficients of scattering and absorption by living bodies, and hence relatively little attenuation. As such, near-infrared light easily passes through living bodies. The high tissue penetration makes infrared light suitable for measurement of in-vivo blood flow dynamics.

A major role of blood is to receive oxygen from the lungs and transport the oxygen to the tissues, and at the same time, receive carbon dioxide from the tissues and circulate the carbon dioxide to the lungs. About 15 g of hemoglobin is present in every 100 ml of blood. Hemoglobin bound to oxygen is called oxyhemoglobin, and hemoglobin not bound to oxygen is called deoxyhemoglobin. The absorption coefficients of these types of hemoglobin have wavelength dependence. Oxyhemoglobin and deoxyhemoglobin exhibit substantially the same absorbance at frequencies near 805 nm. At wavelengths below 805 nm, absorption by deoxyhemoglobin increases. At wavelengths above 805 nm, absorption by oxyhemoglobin increases. These characteristics are exploited to enable measurement or estimation of the respective concentrations of oxyhemoglobin and deoxyhemoglobin.

In traditional NIRS measurement of human skeletal muscle blood flow, a probe of a NIRS apparatus is attached to the skin on a target portion. Infrared light is radiated from a light transmitter of the probe, and as the light passes through the skin, the subcutaneous fat, and the muscle, the light is absorbed and scattered before being detected by a light receiver. Since it is difficult to determine the path length of light detected by NIRS, the amount of hemoglobin to be detected is not an absolute value but a relative value. The intensity of the detected near-infrared light allows for calculation of how much the respective intracellular concentrations of oxyhemoglobin and deoxyhemoglobin have changed from their baseline values. In the following description, oxyhemoglobin or its concentration will be sometimes referred to as Oxy-Hb, and deoxyhemoglobin or its concentration will be sometimes referred to as Deoxy-Hb. The sum of Oxy-Hb and Deoxy-Hb will be sometimes referred to as total hemoglobin or Total-Hb.

When a muscle becomes active due to exercise, the metabolism of muscle cells increases. Oxygen is required for the metabolism of muscle cells. Thus, when a muscle becomes active due to exercise, the consumption of oxygen increases due to metabolism. The decrease of oxygen due to consumption is compensated for by the supply of oxygen in blood. Changes in hemoglobin level measured by NIRS thus reflect the balance between cellular oxygen consumption and oxygen supply. In the early stages of exercise, oxygen consumption exceeds oxygen supply, and Oxy-Hb thus decreases. If balance is maintained between oxygen supply and oxygen consumption during exercise, Oxy-Hb is constant. After exercise, oxygen supply exceeds oxygen consumption, and Oxy-Hb thus increases. As described above, oxygen consumption in active muscle cells (to be herein referred to as “muscle oxygen consumption”) can be calculated or estimated from blood flow dynamics, in particular, Oxy-Hb dynamics.

Unfortunately, existing methods for measuring muscle oxygen consumption by use of NIRS have several issues to be addressed.

First, existing methods measure only the muscle oxygen consumption at a given point in a target portion. Since muscle oxygen consumption varies depending on the muscle site, measurement of muscle oxygen consumption at a given point of a given muscle alone is inadequate for evaluating the effectiveness of strength training or rehabilitation. With such a measurement method, it is difficult to determine a menu for the next effective training based on the measurement results. One way to address this issue would be to place sensors at many locations on the body. It is impractical, however, to perform measurement with many sensors placed on the body before, during, and after exercise.

Second, with existing methods, the behavior of skin blood flow is also reflected in the measurement results, which makes it difficult to measure muscle oxygen consumption with high accuracy. With existing methods, a probe of a NIRS apparatus is attached to the skin on a target portion. Infrared light is radiated from a light transmitter of the probe, and as the light passes through the skin, the subcutaneous fat, and the muscle, the light is absorbed and scattered before being detected by a light receiver. A signal representing the intensity of the detected light thus also contains information about the behavior of skin blood flow. Although it would be conceivable to reduce the influence of skin blood flow by using data representing the distances between the light transmitter and multiple light receivers, this would require use of many light receivers to acquire data for a single point. This makes it further difficult to measure oxygen consumption at multiple locations.

Third, a probe of a NIRS apparatus needs to be attached to the skin on a target portion. Sweating occurs during and after exercise. Thus, keeping the apparatus attached on the skin for a prolonged period of time is not only unhygienic but also makes it difficult to achieve high accuracy measurement due to sweating.

Fourth, it is difficult to compare pre-exercise blood flow dynamics and post-exercise blood flow dynamics. To prevent sweat deposition and interference with exercise execution, it would be conceivable to remove a NIRS apparatus during exercise, and measure and compare pre-exercise blood flow dynamics and post-exercise blood flow dynamics. In that case, the NIRS apparatus needs to be placed at the same location before and after exercise. Unfortunately, such accurate alignment is difficult to achieve, and measurement accuracy tends to be compromised due to placement inconsistency.

The ability to measure exercise-induced changes of blood flow dynamics with the NIRS apparatus and to visualize the effectiveness of muscle exercise would allow for more effective training. Unfortunately, as previously mentioned, existing methods employing NIRS apparatuses have various issues to be addressed. For this reason, evaluating the effectiveness of muscle exercise by use of NIRS apparatuses has not yet become common, although such attempts have been reported at the research level. In particular, no known practical method exists to visualize the effectiveness of muscle exercise.

In view of the foregoing, the inventor herein has investigated a more effective method for visualizing the effectiveness of muscle exercise, and has arrived at embodiments of the present disclosure. Embodiments of the present disclosure make it possible to effectively acquire information on the two-dimensional distribution of muscle oxygen consumption without contact of the measurement apparatus with the skin of the user. This allows for accurate evaluation of the effectiveness of muscle exercise.

An overview of embodiments of the present disclosure will be described below.

A measurement apparatus according to an exemplary embodiment of the present disclosure measures muscle oxygen consumption in a target portion of a user who performs a muscle exercise. The measurement apparatus includes a light source, a sensor, a processing circuit. The sensor includes photoelectric converters. The processing circuit executes a detection operation multiple times to acquire multiple detection signals including a detection signal described below. The detection operation includes the following steps:
(a) causing the light source to emit a light pulse; and
(b) causing the sensor to detect at least part of an internal scattering component of a reflected light pulse, and to output a detection signal representing a spatial distribution of intensity of the at least part of the internal scattering component, the reflected light pulse arising from the target portion due to emission of the light pulse, the internal scattering component being a component scattered in an interior of the target portion. The processing circuit, based on the multiple detection signals, generates and outputs distribution data representing a spatial distribution of muscle oxygen consumption in the target portion.

The term “muscle oxygen consumption” as used herein refers to the amount of oxygen consumed by active muscle cells. The “internal scattering component” may be a component of a reflected light pulse that is detected after the start of decrease in the intensity of the reflected light pulse.

The configuration mentioned above makes it possible to acquire information on the two-dimensional distribution of muscle oxygen consumption without contact of the measurement apparatus with the user’s skin. This makes it possible to accurately evaluate the effectiveness of muscle exercise on a site-by-site basis.

The processing circuit may generate, as the distribution data, image data that represents the spatial distribution of the muscle oxygen consumption in the target portion in a color that varies according to a level of the muscle oxygen consumption. Generating such image data facilitates the user’s recognition of the effectiveness of muscle exercise.

The processing circuit may generate, as the distribution data, image data representing an image, the image including an appearance image and information superimposed on the appearance image, the appearance image being acquired by the sensor or another device and representing an appearance of the user including the target portion, the information representing the spatial distribution of the muscle oxygen consumption. Generating such image data further facilitates the user’s recognition of the effectiveness of muscle exercise.

The processing circuit may: based on the multiple detection signals, estimate a concentration distribution of oxyhemoglobin in blood in the interior of the target portion; and based on time variation of the concentration distribution of the oxyhemoglobin, estimate the spatial distribution of the muscle oxygen consumption. The above-mentioned operation allows for more accurate estimation of the spatial distribution of muscle oxygen consumption due to muscle exercise.

The processing circuit may, based on a slope of time variation of concentration of the oxyhemoglobin, estimate the muscle oxygen consumption. The above-mentioned operation allows for more accurate estimation of the spatial distribution of muscle oxygen consumption due to muscle exercise.

The measurement apparatus may further include a compression unit. The processing circuit may execute the detection operation with blood flow in the target portion being restricted through compression of a part of a body of the user by the compression unit. The compression allows for more accurate estimation of muscle oxygen consumption.

The compression unit may be controlled by the processing circuit. The processing circuit may: before executing the detection operation, cause the compression unit to start compression of the part of the body of the user; and after executing the detection operation, cause the compression unit to end the compression. The above-mentioned operation makes it possible to automate the starting of compression, the executing of detection, and the ending of compression. This leads to improved convenience.

The processing circuit may: execute the detection operation multiple times in each of a first period and a second period, the first period being a period before the user performs the muscle exercise, the second period being a period after the user performs the muscle exercise; and based on the multiple detection signals acquired in the first period and the multiple detection signals acquired in the second period, generate the distribution data. The above-mentioned operation allows for more accurate estimation of the spatial distribution of muscle oxygen consumption due to muscle exercise.

The processing circuit may: acquire data of an appearance image representing an appearance of the user including the target portion, the appearance image being acquired in each of the first period and the second period by the sensor or another device; detect a change in location of the target portion between the first period and the second period, the change in location being detected through matching between one or more features included in the appearance image acquired in the first period, and the one or more features included in the appearance image acquired in the second period; and after applying a process to the multiple detection signals acquired in the first period and to the multiple detection signals acquired in the second period, generate the distribution data, the process compensating for the change in location. The above-mentioned operation allows satisfactory measurement results to be obtained even if there is a change in the location of the target portion between the first period and second period.

The processing circuit may: based on the multiple detection signals acquired in the first period, generate first blood flow data, the first blood flow data representing time course of concentration distribution of oxyhemoglobin in blood in the interior of the target portion; based on the multiple detection signals acquired in the second period, generate second blood flow data, the second blood flow data representing time course of concentration distribution of oxyhemoglobin in blood in the interior of the target portion; and based on the first blood flow data and the second blood flow data, generate the distribution data. The time course of oxyhemoglobin concentration depends on the muscle oxygen consumption due to muscle exercise. Therefore, a comparison of the time course of oxyhemoglobin concentration distribution before and after exercise makes it possible to accurately estimate the spatial distribution of muscle oxygen consumption.

The processing circuit may: based on the first blood flow data, determine a first rate of change, the first rate of change representing a slope of decrease in time variation of concentration of the oxyhemoglobin at multiple points included in the target portion; based on the second blood flow data, determine a second rate of change, the second rate of change representing a slope of decrease in time variation of concentration of the oxyhemoglobin at the multiple points included in the target portion; and based on a difference or ratio between the first rate of change and the second rate of change, estimate the muscle oxygen consumption at the multiple points. The above-mentioned operation allows for more accurate estimation of the spatial distribution of muscle oxygen consumption.

The detection operation in each of the first period and the second period may be performed with blood flow in the target portion being restricted through compression of a part of a body of the user. The processing circuit may: in the first period, fit time variation of concentration of the oxyhemoglobin in a predetermined period to a function, and determine the first rate of change from a time rate of change of the function, the predetermined period being a predetermined period after an increase in concentration of the oxyhemoglobin ends; and in the second period, fit time variation of concentration of the oxyhemoglobin in the predetermined period to the function, and determine the second rate of change from a time rate of change of the function. The above-mentioned operation allows for more accurate estimation of the spatial distribution of muscle oxygen consumption.

The processing circuit may: in response to the second rate of change being greater than or equal to a factor “a” of the first rate of change, add, to the distribution data to be output, information indicating that the muscle oxygen consumption is relatively large, the factor “a” being a real number greater than one; and in response to the second rate of change being less than the factor “a” of the first rate of change, add, to the distribution data to be output, information indicating that the muscle oxygen consumption is relatively small. The above-mentioned operation makes it possible to, for example, cause a display that has acquired the distribution data to display an image indicating at which point the muscle oxygen consumption is relatively large.

The processing circuit may: based on the information indicating that the muscle oxygen consumption is relatively large, determine a first region in the target portion, the first region being a region where the muscle oxygen consumption is relatively large; and based on the information indicating that the muscle oxygen consumption is relatively small, determine a second region in the target portion, the second region being a region where the muscle oxygen consumption is relatively small. The distribution data may include an image that highlights the first region or the second region. Display of such an image allows the user to easily recognize the effectiveness of muscle exercise for each individual point in the target portion.

The processing circuit may further generate and output data representing a training plan used to train a muscle in a region where the muscle oxygen consumption is relatively small, the region being included in the target portion. This allows the user to, based on such data, learn a training plan for effectively training a muscle site for which low effectiveness of muscle exercise is observed.

The processing circuit may: acquire history data representing information on the muscle exercise performed by the user; and based on the history data, adjust the training plan. The above-mentioned operation makes it possible to present the user with a more suitable training plan.

The processing circuit may: acquire identification data that identifies the user; and based on the identification data, adjust the training plan. This makes it possible to present an optimum training plan for each individual user.

The light source may emit a first light pulse and a second light pulse, the first light pulse having a first wavelength of greater than or equal to 650 nm and less than 805 nm, the second light pulse having a second wavelength of greater than or equal to 805 nm and less than or equal to 950 nm. The detection operation may include: causing the light source to emit the first light pulse; causing the sensor to detect at least part of a first internal scattering component of a first reflected light pulse, and to output a first detection signal representing a spatial distribution of intensity of the at least part of the first internal scattering component, the first reflected light pulse arising from the target portion due to emission of the first light pulse, the first internal scattering component being a component scattered in the interior of the target portion; causing the light source to emit the second light pulse; and causing the sensor to detect at least part of a second internal scattering component of a second reflected light pulse, and to output a second detection signal representing a spatial distribution of intensity of the at least part of the second internal scattering component, the second reflected light pulse arising from the target portion due to emission of the second light pulse, the second internal scattering component being a component scattered in the interior of the target portion. The processing circuit may: based on the first detection signal and the second detection signal, estimate a concentration distribution of oxyhemoglobin in blood in the interior of the target portion; and based on time variation of the concentration distribution of the oxyhemoglobin, estimate the spatial distribution of the muscle oxygen consumption. The above-mentioned operation allows for more accurate estimation of the spatial distribution of muscle oxygen consumption.

The measurement apparatus may further include an augmented reality (AR) glass with a transparent display. The transparent display may display a distribution image representing the distribution data, the distribution image being superimposed on an appearance of the user that is visible through the transparent display. This allows the user to easily recognize at which site training has been highly effective.

A method according to another embodiment of the present disclosure is executable by a computer. The computer is included in a measurement apparatus that measures muscle oxygen consumption in a target portion of a user who performs a muscle exercise. The method includes: executing a detection operation multiple times to acquire multiple detection signals, the detection operation including causing a light source to emit a light pulse, and causing a sensor to detect at least part of an internal scattering component of a reflected light pulse, and to output a detection signal representing a spatial distribution of intensity of the at least part of the internal scattering component, the sensor including photoelectric converters, the reflected light pulse arising from the target portion due to emission of the light pulse, the internal scattering component being a component scattered in an interior of the target portion, the detection signal being included in the multiple detection signals; and based on the multiple detection signals, generating and outputting distribution data, the distribution data representing a spatial distribution of muscle oxygen consumption in the target portion.

A non-transitory computer-readable recording medium according to another embodiment of the present disclosure stores a computer program executable by a computer. The computer is included in a measurement apparatus that measures muscle oxygen consumption in a target portion of a user who performs a muscle exercise. The computer program causes the computer to execute a process. The process includes: executing a detection operation multiple times to acquire multiple detection signals, the detection operation including causing a light source to emit a light pulse, and causing a sensor to detect at least part of an internal scattering component of a reflected light pulse, and to output a detection signal representing a spatial distribution of intensity of the at least part of the internal scattering component, the sensor including photoelectric converters, the reflected light pulse arising from the target portion due to emission of the light pulse, the internal scattering component being a component scattered in an interior of the target portion, the detection signal being included in the multiple detection signals; and based on the multiple detection signals, generating and outputting distribution data, the distribution data representing a spatial distribution of muscle oxygen consumption in the target portion.

Embodiments of the present disclosure will be described below in more detail with reference to the attached drawings.

### Embodiment

### 1. Configuration

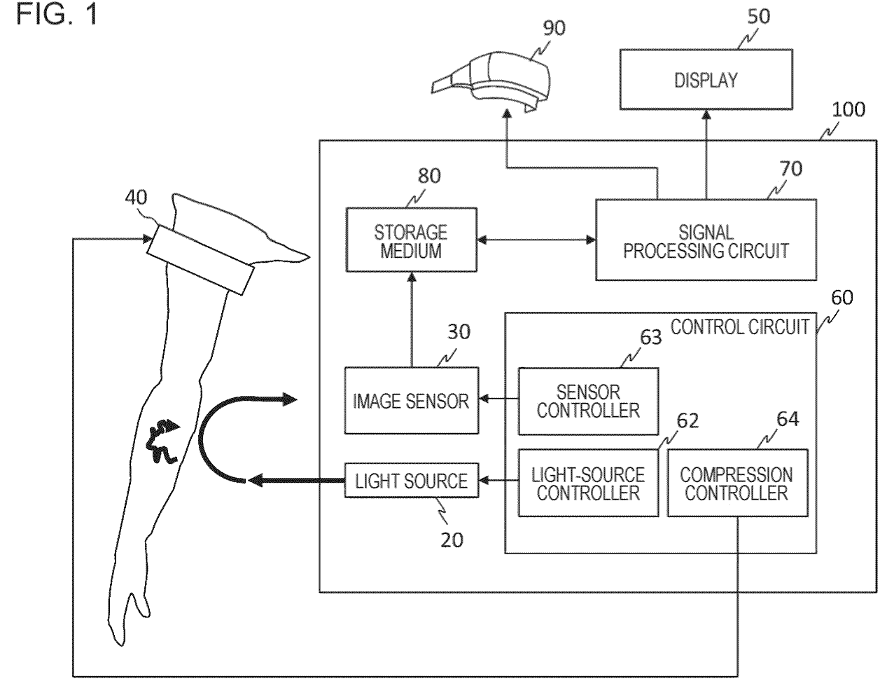

FIG. 1 schematically illustrates the configuration of a system for measuring muscle oxygen consumption according to an exemplary embodiment of the present disclosure. The system includes a measurement apparatus 100, a compression unit 40, a display 50, and an AR glass 90. The measurement apparatus 100 includes a light source 20, an image sensor 30, a control circuit 60, a signal processing circuit 70, and a storage medium 80 such as a memory. FIG. 1 also depicts an arm of a user who uses the measurement apparatus 100. According to the embodiment, the control circuit 60 and the signal processing circuit 70 serve as the “processing circuit” mentioned above.

The compression unit 40 is to be placed on the user to compress a part of the user’s body to temporarily occlude blood flow to the part of the body. According to the embodiment, the compression unit 40 is placed on a user’s arm. The compression unit 40 is placed at different sites depending on the location of the target portion. For example, to measure muscle oxygen consumption of a leg, the compression unit 40 may be placed on the leg. To measure muscle oxygen consumption of the trunk, the compression unit 40 may be placed on the torso.

The light source 20 emits a light pulse for irradiating a target portion of the user. The light source 20 includes one or more light-emitting elements, and emits a light pulse toward the target portion. The light source 20 may be capable of emitting two different wavelengths of light pulses. For example, the light source 20 may be capable of emitting a first light pulse with a first wavelength of greater than or equal to 650 nm and less than 805 nm, and a second light pulse with a second wavelength of greater than or equal to 805 nm and less than or equal to 950 nm.

The image sensor 30 detects at least part of a light pulse returning from the user’s body, and outputs a detection signal representative of the detection result. The image sensor 30 includes a two-dimensional array of photodetection cells. Each photodetection cell includes a photoelectric converter, and outputs an electrical signal according to the quantity of received light. Although the embodiment employs the image sensor 30, other types of sensors with a two-dimensional array of photoelectric converters may be employed.

The control circuit 60 controls the compression unit 40, the light source 20, and the image sensor 30. The control circuit 60 includes a compression controller 64, a light-source controller 62, and a sensor controller 63. The compression controller 64 controls the compression unit 40. The light-source controller 62 controls the light source. The sensor controller 63 controls the image sensor 30.

The compression controller 64 controls operation of the compression unit 40. For example, the compression controller 64 controls one or both of pressure and the timing of compression.

The light-source controller 62 controls the emission of light by the light source 20. For example, the light-source controller 62 controls at least one of the intensity, pulse duration, emission timing, or wavelength of a light pulse emitted from the light source 20.

The sensor controller 63 controls the timing of signal storage in each photodetection cell of the image sensor 30.

The compression controller 64, the light-source controller 62, and the sensor controller 63 may be implemented by three discrete circuits, or may be implemented by a single circuit. The compression controller 64, the light-source controller 62, and the sensor controller 63 may be implemented by the control circuit 60 executing a control program stored in a memory (not illustrated).

The signal processing circuit 70 processes a detection signal output from the image sensor 30. The signal processing circuit 70 generates, based on the detection signal, information representing the state of blood flow in the target portion of the user (to be referred to as “blood flow information” hereinafter). The blood flow information may, for example, include at least one selected from the group consisting of: blood flow; oxyhemoglobin concentration in blood; deoxyhemoglobin concentration in blood; total hemoglobin concentration as the sum of oxyhemoglobin concentration and deoxyhemoglobin concentration; and blood oxygen saturation. Based on the time course of blood flow information on the target portion, the signal processing circuit 70 generates and outputs distribution data representing the two-dimensional distribution of muscle oxygen consumption.

Although the control circuit 60 and the signal processing circuit 70 are discrete circuits according to the embodiment, the functions of these circuits may be implemented by a single electronic circuit.

The display 50 displays an image based on distribution data generated by the signal processing circuit 70. For example, the display 50 displays an image including an appearance image representing the user’s appearance, and information superimposed on the appearance image and representing the spatial distribution of muscle oxygen consumption. The display 50 may be, for example, any display such as a liquid crystal display or an organic EL display. The display 50 may be built in the measurement apparatus 100.

The AR glass 90 includes a transparent display. The AR glass 90 is capable of displaying, on the transparent display, an image based on distribution data generated by the signal processing circuit 70. For example, the AR glass 90 displays, on the transparent display, an image based on distribution data in such a way that the image is superimposed on the user’s appearance that is visible through the transparent display.

Configurations of individual components will be described below in more specific detail.

### 1-1. Compression Unit 40

The compression unit 40 compresses a part of the user’s body. Due to the compression, blood flow to the part of the user’s body is occluded for a predetermined period of time. The compression unit 40 may include, for example, a cuff. The compression may be performed, for example, with the cuff being wrapped around the base of an arm or leg. The compression pressure to be applied may be set to, for example, a predetermined value such as 40 mmHg or 200 mmHg. The compression causes temporary occlusion of venous or arterial blood flow. The duration of the compression is set to a predetermined time, for example, one minute. The compression unit 40 may be connected by wire or wirelessly with the control circuit 60. The compression unit 40 operates in accordance with a control signal input from the control circuit 60. The compression unit 40 is not necessarily connected with the control circuit 60 but may be manually operated. 1-2. Light Source 20

The light source 20 includes one or more light-emitting elements. Each light-emitting element may include, for example, a laser diode that emits laser light. The light source 20 emits a light pulse in accordance with a control signal input from the light-source controller 62 of the control circuit 60.

The light source 20 is positioned to emit light toward a target portion of the user. The target portion is, for example, the arm or the leg of the user. More specifically, the target portion may be the upper arm, the forearm, the thigh, or the lower leg of the user. Examples of the target portions are not limited to the above-mentioned sites. For example, the target portion may be the abdomen, the chest, the lower back, the upper back, or the shoulder.

Light arriving at a target portion of the user after being emitted from the light source 20 is divided into two components, one reflected at the surface of the target portion and the other scattered in the interior of the target portion. Herein, the component reflected at the surface will be referred to as “surface reflection component,” and the component scattered in the interior will be referred to as “internal scattering component.” The internal scattering component is a component that is reflected or scattered once, or scattered multiple times, in the interior of the target portion. For light emitted toward the user’s arm or leg, its internal scattering component reaches a depth of about 8 mm to 16 mm from the skin surface of the arm or leg. That is, the internal scattering component is a component of light that passes through the skin and subcutaneous fat to reach the underneath muscle, where the component is scattered before going back toward the measurement apparatus 100 again. The surface reflection component includes the three following sub-components: a regular reflection component, a diffuse reflection component, and a scattered reflection component. The regular reflection component refers to a reflection component whose angle of reflection is equal to the angle of incidence. The diffuse reflection component refers to a component that is diffused and reflected by irregularities present on the surface. The scattered reflection component refers to a component that is scattered and reflected by the internal tissues near the surface. For light emitted toward the user’s arm or leg, its scattered reflection component is a component that is scattered and reflected inside the epidermis of the leg or arm. The following description assumes that the surface reflection component reflected at the surface of the target portion includes these three sub-components. The following description also assumes that the internal scattering component does not include a component that is scattered and reflected by the internal tissues near the surface. Each of the surface reflection component and the internal scattering component changes its direction of travel when reflected or scattered, and part of the component reaches the image sensor 30. The surface reflection component may contain surface information on the target portion, for example, epidermal blood flow information. The internal scattering component may contain internal information on the target portion, for example, information on the blood flow near the muscle tissue. Accordingly, detecting the surface reflection component makes it possible to acquire surface information on the target portion, for example, epidermal blood flow information. Detecting the internal scattering component makes it possible to acquire internal information on the target portion, for example, information on the blood flow near the muscle tissue.

According to the embodiment, for light bouncing back from a target portion such as the user’s arm or leg (to be herein referred to as “reflected light”), at least part of at least its internal scattering component is detected. The internal scattering component varies in intensity as a function of the activity of the user’s muscle tissue. Accordingly, analyzing the time course of the internal scattering component makes it possible to estimate the state of the user’s muscular activity.

Reference will now be made to an exemplary method for detecting the internal scattering component. In accordance with an instruction from the control circuit 60, the light source 20 repeatedly emits a light pulse at predetermined time intervals or at predetermined times. The light pulse to be emitted from the light source 20 may be, for example, a rectangular wave with a nearly zero falling period. As used herein, the “falling period” of a light pulse refers to a period from when a decrease in the intensity of the light pulse begins to when the decrease ends. Typically, rays of light entering a target portion of the user propagate along various paths in the interior of the target portion, and emerge from the target portion with time differences. The internal scattering component of a light pulse thus has a broadened trailing edge. If the target portion is an arm or leg, the broadening of the trailing edge of the internal scattering component has a duration of about 4 ns. With this in mind, the duration of the falling period of a light pulse emitted by the light source 20 may be set to, for example, less than or equal to half the duration of the broadening, that is, to less than or equal to 2 ns. The duration of the falling period may be further set to less than or equal to half the above duration, that is, to 1 ns. The rising period of a light pulse emitted from the light source 20 may have any duration. As used herein, the “rising period” of a light pulse refers to a period from when an increase in the intensity of the light pulse begins to when the increase ends. For detection of the internal scattering component according to the embodiment, the falling part of the light pulse is used, and the rising part is not used. The rising part of the light pulse may be used for detection of the surface reflection component. The light source 20 may include, for example, a laser such as an LD. Light emitted from the laser has sharp time response characteristics with the falling part of the light pulse being at substantially right angles to the time axis.

The light source 20 may emit a single wavelength of light, or may emit multiple wavelengths of light. The multiple wavelengths of light may be emitted by different light-emitting elements. If two different wavelengths of light are to be emitted from two light-emitting elements, the two wavelengths of light may be designed to pass through a target portion of the user and return to the image sensor 30 after travelling substantially the same optical path length. For example, the two light-emitting elements may be positioned in such a way that the distance between the image sensor 30 and one of the light-emitting elements, and the distance between the image sensor 30 and the other light-emitting element are the same, and that the light-emitting elements are in rotational symmetry about the center of the image sensor 30.

The light to be emitted from the light source 20 may have, for example, any wavelength within a wavelength range of greater than or equal to 650 nm and less than or equal to 950 nm. This wavelength range falls within the red to near-infrared wavelength range. The wavelength range mentioned above is called “biological window,” where there is relatively less absorption of light by water within a living body and by the skin. For measurement with a living body as a subject, use of the above-mentioned wavelength range allows for increased detection sensitivity. It is presumed that in detecting a change in user’s blood flow as in the embodiment, light used for the detection is mainly absorbed by oxyhemoglobin and deoxyhemoglobin. A change in blood flow generally causes a change in oxyhemoglobin concentration and a change in deoxyhemoglobin concentration. Such changes also cause a change in the degree of light absorption. Therefore, a change in blood flow also causes a temporal change in the quantity of detected light.

Oxyhemoglobin and deoxyhemoglobin differ in the wavelength dependence of light absorption. At wavelengths greater than or equal to 650 nm and less than 805 nm, deoxyhemoglobin has a coefficient of light absorption that is greater than the coefficient of light absorption by oxyhemoglobin. At the wavelength of 805 nm, deoxyhemoglobin and oxyhemoglobin have substantially the same coefficient of light absorption. At wavelengths greater than 805 nm and less than or equal to 950 nm, oxyhemoglobin has a coefficient of light absorption that is greater than the coefficient of light absorption by deoxyhemoglobin.

Accordingly, the light source 20 may be capable of emitting a first light pulse with a wavelength of greater than or equal to 650 nm and less than 805 nm, and a second light pulse with a wavelength of greater than or equal to 805 nm and less than or equal to 950 nm. Irradiation of the target portion with the first light pulse and the second light pulse that differ in wavelength makes it possible to estimate the respective concentrations of oxyhemoglobin and deoxyhemoglobin that are contained in blood inside the target portion. Irradiating the target portion with two light pulses of different wavelengths in this way allows for acquisition of more detailed internal information on the target portion.

According to the embodiment, blood flow inside the user’s muscle is measured in a non-contact manner. In view of the possibility that light emitted from the light source 20 may impinge on the user’s eyes, the light source 20 to be used may be designed by taking the effect on the user’s retina into consideration. For example, the light source 20 may be designed to satisfy the requirements for Class 1 of the laser safety standard developed in individual countries. If the requirements for Class 1 are satisfied, the user is irradiated with light with such a low illuminance that the Accessible Emission Limit (AEL) is below 1 mW. The light source 20 itself does not have to satisfy the requirements for Class 1. For example, a diffuser or a ND filter may be installed in front of the light source 20 to diffuse or attenuate light so that the requirements for Class 1 of the laser safety standard are satisfied.

In irradiating a target portion of the user with light to measure blood flow near the muscle cell, the quantity of the internal scattering component of the light can have a very small value, which is about several thousandths to several ten thousandths of that of the surface reflection component. With the laser safety standard further taken into account, the quantity of light that can be radiated is very small. This makes detection of the internal scattering component very difficult. Even for such cases, making the light source 20 emit a light pulse with a relatively long pulse duration makes it possible to increase the integrated quantity of the internal scattering component having a relative time delay. This helps to increase the quantity of detected light, and improve the SN ratio.

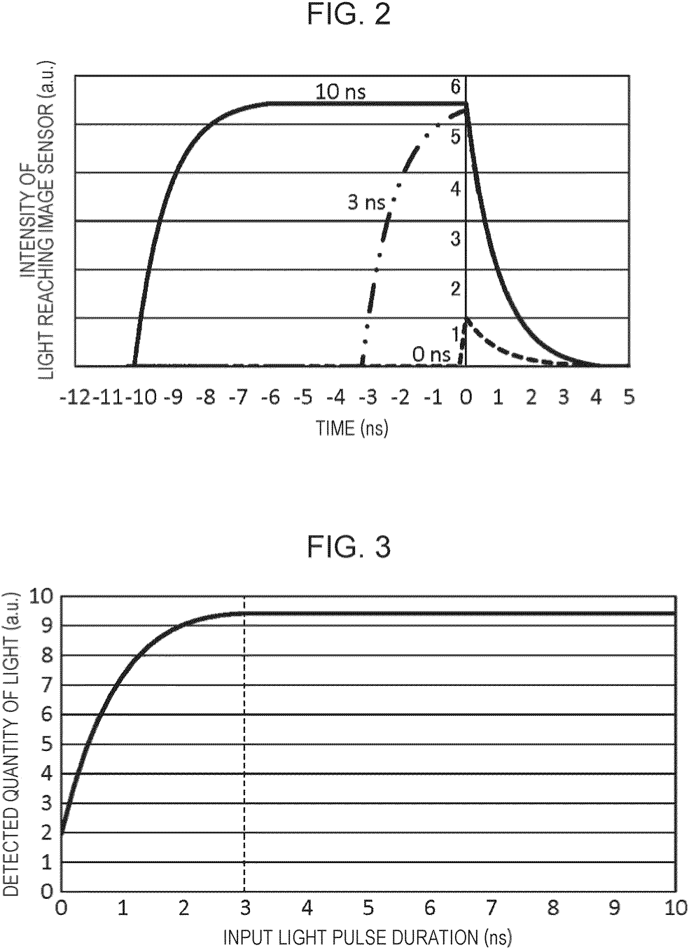

The light source 20 emits, for example, a light pulse with a pulse duration of greater than or equal to 3 ns. Generally, light scattered in living tissue such as muscle has a temporal broadening of about 4 ns. FIG. 2 illustrates an example of the time variation of the intensity of light arriving at the image sensor 30. FIG. 2 illustrates exemplary light pulses emitted from the light source 20 that have three different durations of 0 ns, 3 ns, and 10 ns. As illustrated in FIG. 2, the greater the duration of a light pulse emitted from the light source 20, the greater the quantity of the internal scattering component appearing at the trailing edge of the light pulse that travels to reach the image sensor 30 from the user.

FIG. 3 is an illustration with the horizontal axis representing the duration of an input light pulse, which is a light pulse emitted from the light source 20, and the vertical axis representing the quantity of light detected by the image sensor 30. The image sensor 30 includes an electronic shutter. The results in FIG. 3 are obtained under the condition of the electronic shutter being opened after elapse of 1 ns from the time of arrival, at the image sensor 30, of the trailing edge of the light pulse reflected off the surface of the target portion. This condition is selected because immediately after the arrival of the trailing edge of the light pulse, the proportion of the surface reflection component is large relative to that of the internal scattering component. As illustrated in FIG. 3, the quantity of detected light can be maximized by making the duration of the light pulse emitted from the light source 20 greater than or equal to 3 ns.

The light source 20 may emit a light pulse with a pulse duration of greater than or equal to 5 ns, or with an even longer pulse duration of greater than or equal to 10 ns. An excessively long pulse duration, however, leads to waste as the quantity of unused light increases. Accordingly, the light source 20 may, for example, emit a light pulse with a pulse duration of less than or equal to 50 ns. Alternatively, the light source 20 may emit a light pulse with a pulse duration of less than or equal to 30 ns, or with an even shorter pulse duration of less than or equal to 20 ns. If the rectangular pulse has a duration of several ns to several tends of ns, the light source 20 can be driven at a comparatively low voltage. This makes it possible to reduce the size and cost of the measurement apparatus 100.

The radiation pattern of the light source may be, for example, a pattern that provides a uniform intensity distribution within a region to be irradiated. The embodiment differs from existing methods in this respect. With existing methods, the detector and the light source are placed about 3 cm apart so that the surface reflection component is spatially separated from the internal scattering component. This necessarily results in discrete radiation of light. By contrast, the measurement apparatus 100 according to the embodiment allows for temporal separation of the surface reflection component from the internal scattering component to thereby reduce the surface reflection component. This in turn allows use of the light source 20 with a radiation pattern that provides a uniform intensity distribution. Alternatively, such a radiation pattern with a uniform intensity distribution may be produced by use of a diffuser that diffuses light emitted from the light source 20.

According to the embodiment, the internal scattering component can be detected even directly under the irradiation point on the target portion. The embodiment also makes it possible to enhance measurement resolution by irradiating the target portion with light over a spatially large area.

### 1-3. Image Sensor 30

The image sensor 30 may be, for example, any imager such as a CCD image sensor or a CMOS image sensor. The image sensor 30 includes a two-dimensional array of photodetection cells on its light-receiving surface. Each photodetection cell may include, for example, a photoelectric converter such as a photodiode, and one or more charge storage sections. The photoelectric converter generates, through photoelectric conversion, a signal charge corresponding to the quantity of received light. Each charge storage section stores the signal charge generated by the photoelectric converter. The image sensor 30 is thus capable of acquiring two-dimensional information on the user at once. A photodetection cell will be herein sometimes referred to as “pixel.”

The image sensor 30 may include an electronic shutter. The electronic shutter is a circuit that controls the timing of imaging. According to the embodiment, the sensor controller 63 of the control circuit 60 serves as the electronic shutter. The electronic shutter controls a period of single signal storage in which received light is converted into an effective electrical signal and stored, and a period in which signal storage is stopped. The signal storage period will be also referred to as “exposure period.” In the following description, the duration of the exposure period will be also referred to as “shutter duration.” The period of time from when one exposure period ends to when the next exposure period begins will be also referred to as “non-exposure period.” Hereinafter, a state in which exposure is being performed will be sometimes referred to as “OPEN,” and a state in which exposure is being stopped will be sometimes referred to as “CLOSE.”

The image sensor 30 allows the exposure period and the non-exposure period to be adjusted by means of the electronic shutter in a sub-nanosecond range, for example, in the range of 30 ps to 1 ns. TOF cameras, which are used for distance measurement, detect all of light emitted from a light source and reflected back from a subject. Thus, for such a TOF camera, the shutter duration needs to be greater than the pulse duration of light. By contrast, the measurement apparatus 100 according to the embodiment does not require the shutter duration to be greater than the pulse duration. The shutter duration can be set to a value of, for example, greater than or equal to 1 ns and less than or equal to 30 ns. The measurement apparatus 100 according to the embodiment makes it possible to reduce the shutter duration, and consequently reduce the influence of dark current included in a detection signal.

In irradiating a target portion of the user such as the arm or leg with light to detect information such as blood flow, the light undergoes a very large in-vivo attenuation. For example, the light exiting the target portion may be attenuated in intensity to about one millionth of the light entering the target portion. Thus, in some cases, radiation of a single pulse alone may not provide a sufficient quantity of light for detecting the internal scattering component. For radiation according to Class 1 of the laser safety standard, in particular, the quantity of light provided is very small. In this case, by executing emission of a light pulse by the light source 20 multiple times, and likewise executing exposure of the image sensor 30 by the electronic shutter multiple times, the detection signal can be integrated for improved sensitivity.

Reference will be made below to an example in which each pixel of the image sensor 30 includes a photoelectric converter such as a photodiode, and charge storage sections. The charge storage sections in each pixel may include a charge storage section for storing a signal charge generated by the surface reflection component of a light pulse, and a charge storage section for storing a signal charge generated by the internal scattering component of the light pulse. The control circuit 60 causes the image sensor 30 to detect the surface reflection component of a reflected light pulse reflected back from a target portion of the user, by causing the image sensor 30 to detect a portion of the pulse corresponding to a period before the start of falling of the pulse. The control circuit 60 causes the image sensor 30 to detect the internal scattering component of the light pulse reflected back from the target portion of the user, by causing the image sensor 30 to detect a portion of the pulse corresponding to a period after the start of falling of the pulse. The light source 20 in the present example emits two wavelengths of light.

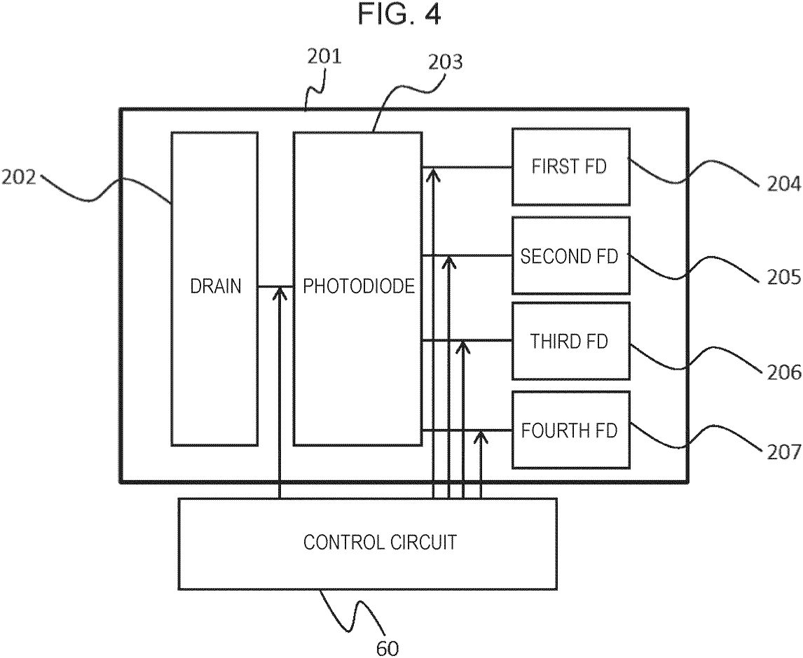

FIG. 4 illustrates an exemplary schematic configuration of one pixel 201 of the image sensor 30. It is to be noted that FIG. 4 schematically represents the configuration of one pixel 201, and does not necessarily reflect the actual configuration. The pixel 201 in this example includes the following components: a photodiode 203 capable of photoelectric conversion; a first floating diffusion (FD) 204, a second floating diffusion 205, a third floating diffusion 206, and a fourth floating diffusion 207, which are charge storage sections; and a drain 202 for discharging signal charge.

Photons incident on individual pixels due to a single emission of a light pulse are converted by the photodiode 203 into signal electrons serving as signal charge. In accordance with a control signal input to the image sensor 30 from the control circuit 60, the resulting signal electrons are either discharged to the drain 202, or distributed to one of the first to fourth floating diffusions 204 to 207.

The emission of a light pulse from the light source 20, the storage of signal charge to the first floating diffusion 204, the second floating diffusion 205, the third floating diffusion 206, and the fourth floating diffusion 207, and discharge of signal charge to the drain 202 are performed repeatedly in this order. The repetition is fast, and can occur, for example, several ten thousand times to several hundred million times within the duration of one frame of a moving image. The duration of one frame is, for example, approximately 1/30 seconds. The pixel 201 eventually generates and outputs four image signals, each based on the signal charge stored in the corresponding one of the first to four floating diffusions 204 to 207.

The control circuit 60 in the present example causes the light source 20 to repeatedly emit, in a sequential manner, a first light pulse having a first wavelength, and a second light pulse having a second wavelength. As the first and second wavelengths, two wavelengths with different coefficients of absorption by the internal tissues of a target portion are selected to enable analysis of the internal condition of the target portion. For example, a wavelength of less than 805 nm may be selected as the first wavelength, and a wavelength of greater than or equal to 805 nm may be selected as the second wavelength. This makes it possible to detect changes in the respective concentrations of oxyhemoglobin and deoxyhemoglobin in blood.

The control circuit 60 first causes the light source 20 to emit the first light pulse. The control circuit 60 causes a signal charge to be stored in the first floating diffusion 204 in a first period during which the surface reflection component of the first light pulse is incident on the photodiode 203. The control circuit 60 then causes a signal charge to be stored in the second floating diffusion 205 in a second period during which the internal scattering component of the first light pulse is incident on the photodiode 203. Subsequently, the control circuit 60 causes the light source 20 to emit the second light pulse. The control circuit 60 causes a signal charge to be stored in the third floating diffusion 206 in a third period during which the surface reflection component of the second light pulse is incident on the photodiode 203. The control circuit 60 then causes a signal charge to be stored in the fourth floating diffusion 207 in a fourth period during which the internal scattering component of the second light pulse is incident on the photodiode 203.

In this way, after emission of the first light pulse is initiated, the control circuit 60 causes the signal charge from the photodiode 203 to be sequentially stored, with a predetermined time difference, into each of the first floating diffusion 204 and the second floating diffusion 205. Thereafter, after emission of the second light pulse is initiated, the control circuit 60 causes the signal charge from the photodiode 203 to be sequentially stored, with the predetermined time difference, into each of the third floating diffusion 206 and the fourth floating diffusion 207. The above-mentioned operation is repeated multiple times.

To estimate the quantities of disturbance light and ambient light, a period may be provided during which signal charge is stored in another floating diffusion (not illustrated) with the light source 20 being turned off. A signal with the disturbance light and ambient light components removed can be obtained by subtracting the signal charge stored in the other floating diffusion from the signal charge stored in each of the first to fourth floating diffusions 204 to 207.

Although in the embodiment each pixel includes four charge storage sections, each pixel may, depending on the intended purpose, be designed to include any number of charge storage sections greater than or equal to one. For example, if the surface reflection component and the internal scattering component are to be detected by use of only a single wavelength, each pixel may include two charge storage sections. If a single wavelength is to be used, and the surface reflection component is not to be detected, each pixel may include a single charge storage section. If only the internal scattering component is to be detected by use of two wavelengths, each pixel may include two charge storage sections. Even in cases involving use of two or more wavelengths, if imaging at each wavelength is performed in a different frame, then each pixel may include a single charge storage. Likewise, even in cases involving detection of both the surface reflection component and the internal scattering component, if these two components are to detected in different frames, then each pixel may include a single charge storage section.

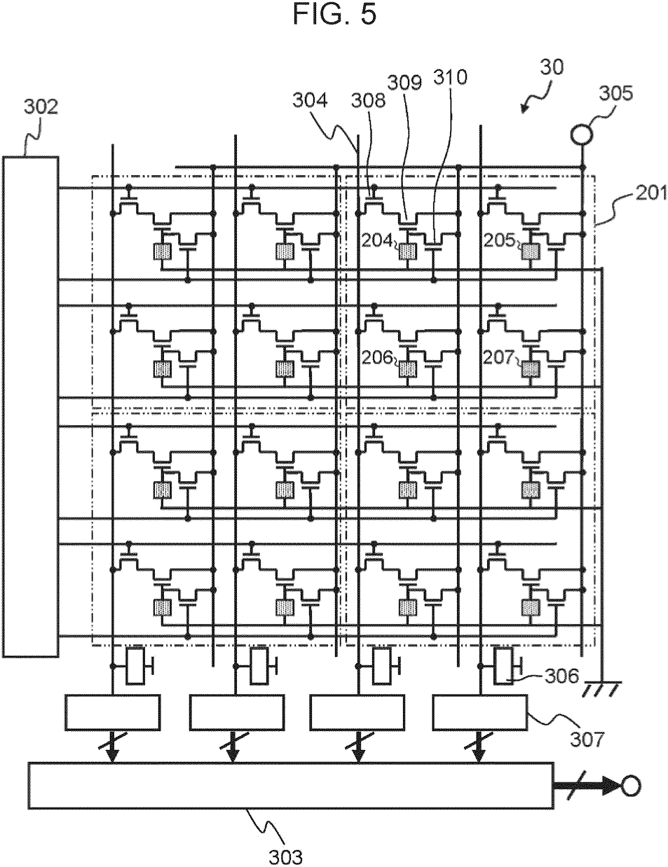

FIG. 5 illustrates an exemplary configuration of the image sensor 30. In FIG. 5, regions bounded by two-dot chain lines each correspond to a single pixel 201. The pixel 201 includes a single photodiode. Although FIG. 5 depicts only four pixels arranged in two rows and two columns, more pixels may be arranged in practice. The pixel 201 includes the first to fourth floating diffusions 204 to 207. Signals stored in the first to fourth floating diffusions 204 to 207 are treated as if the signals of four pixels of a typical CMOS image sensor, and are output from the image sensor 30.

Each pixel 201 includes four signal detection circuits. Each signal detection circuit includes a source follower transistor 309, a row selection transistor 308, and a reset transistor 310. In the present example, the reset transistor 310 corresponds to the drain 202 illustrated in FIG. 4. The gate of the reset transistor 310 receives input of a drain discharge pulse. A non-limiting example of each of these transistors may be a field-effect transistor formed on a semiconductor substrate. As illustrated in FIG. 5, one of the input terminal and the output terminal of the source follower transistor 309, and one of the input terminal and the output terminal of the row selection transistor 308 are connected with each other. The one of the input terminal and the output terminal of the source follower transistor 309 is typically a source. The one of the input terminal and the output terminal of the row selection transistor 308 is typically a drain. The gate of the source follower transistor 309, which serves as a control terminal, is connected with the photodiode 203. The signal charge of holes or electrons generated by the photodiode 203 is stored in a floating diffusion, which is a charge storage section located between the photodiode 203 and the source follower transistor 309.

Although not illustrated in FIG. 5, the photodiode 203 is connected with the first to fourth floating diffusions 204 to 207. One or more switches may be disposed between the photodiode 203 and each of the first to fourth floating diffusions 204 to 207. The switch is designed to, in accordance with a charge storage pulse from the control circuit 60, switch the states of conduction between the photodiode 203 and each of the first to fourth floating diffusions 204 to 207. This controls when to start and stop the storage of signal charge for each of the first to fourth floating diffusions 204 to 207. The electronic shutter according to the embodiment includes a mechanism for such exposure control.

The signal charge stored in each of the first to fourth floating diffusions 204 to 207 is read out as the gate of the row selection transistor 308 is turned ON by a row selection circuit 302. At this time, the current flowing from a source follower power supply 305 into each of the source follower transistor 309 and a source follower load 306 is amplified in accordance with the signal potential of each of the first to fourth floating diffusions 204 to 207. An analog signal due to the current is read out from each vertical signal line 304, and converted into digital signal data by an analog-to-digital (AD) conversion circuit 307 connected for each individual column. The digital signal data is read out column by column by a column selection circuit 303 for output from the image sensor 30. The row selection circuit 302 and the column selection circuit 303 each first read out one row, and then read out the next row. Thereafter, the row selection circuit 302 and the column selection circuit 303 likewise read out information on the signal charge in each of the floating diffusions in all rows. After all signal charges are read out, the control circuit 60 turns on the gate of each reset transistor 310 to thereby reset all floating diffusions. This completes imaging of one frame. Thereafter, high-speed frame imaging is repeated similarly to complete imaging of a series of frames by the image sensor 30.

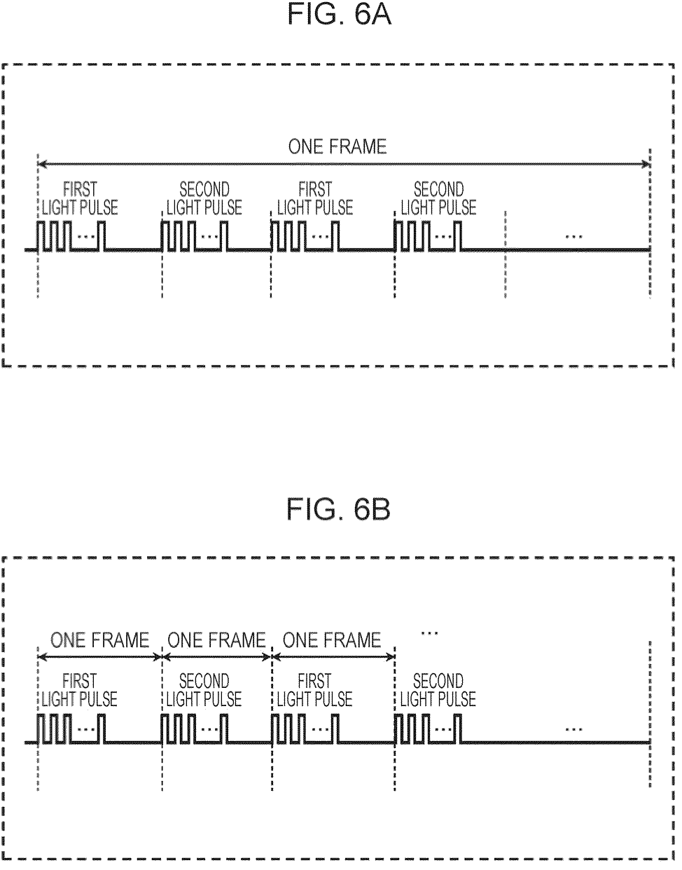

FIG. 6A illustrates an example of operation within a single frame. As illustrated in FIG. 6A, emission of the first light pulse, and emission of the second light pulse may be switched alternately multiple times within a single frame. This leads to reduced time difference between the acquisitions of detection signals at two different wavelengths. As a result, even in the presence of motion of the user, imaging with the first light pulse, and imaging with the second light pulse can be performed in a substantially simultaneous manner.

FIG. 6B illustrates another exemplary detection operation performed by using two wavelengths of light. As illustrated in FIG. 6B, detection of a reflected light pulse due to the first light pulse, and detection of a reflected light pulse due to the second light pulse may be switched on a frame-by-frame basis. This is accomplished by, for example, switching the emission of the first light pulse and the emission of the second light pulse on a frame-by-frame basis. In that case, each pixel 201 may include a single charge storage section. Such a configuration helps to reduce the number of charge storage sections in each pixel 201. This allows for increased size of each pixel 201, and improved sensitivity.

The light source 20 may emit a single wavelength of light. Even in that case, the state of muscular activity can be roughly estimated.

Although the foregoing description of the embodiment is directed to an example of the image sensor 30 being a CMOS image sensor, the image sensor 30 to be used may be another type of imager. The image sensor 30 may be, for example, any one of the following types of imagers: a CCD image sensor; a single-photon counter; and an amplifying image sensor such as an EMCCD or ICCD. Instead of the image sensor 30 with a two-dimensional array of photodetection cells, sensors each including a single photoelectric converter may be used. Even if a two-dimensional array of single-pixel sensors is used, this allows for generation of two-dimensional data on a target portion.

According to the embodiment, the image sensor 30 is capable of detecting the surface reflection component and/or internal scattering component of a light pulse. Temporal or spatial variation of the surface reflection component allows for acquisition of first biological information such as the user’s pulse rate. Temporal or spatial variation of the internal scattering component allows for acquisition of second biological information such as the user’s muscle oxygen consumption.

The first biological information may be acquired by a method different from the method for detecting the surface reflection component. The first biological information does not have to be acquired in the first place. For example, the first biological information may be acquired by use of another type of detector different from the image sensor 30. In that case, the image sensor 30 may detect only the internal scattering component. Such another type of detector may be, for example, a radar or a thermograph. The first biological information may be, for example, at least one selected from the group consisting of the pulse rate, perspiration, breathing, and body temperature of the user. The first biological information is biological information other than muscle oxygen consumption information, the muscle oxygen consumption information being obtained by detection of the internal scattering component of a light pulse radiated to a target portion of the user. The expression “biological information other than muscle oxygen consumption information” does not mean that the first biological information contains absolutely no information due to muscle oxygen consumption activity. The first biological information may contain biological information due to biological activity different from muscle oxygen consumption activity. The first biological information may be, for example, biological information due to autonomous or reflexive biological activity.

### 1-4. Control Circuit 60 and Signal Processing Circuit 70

The control circuit 60 adjusts the time difference between the timing of emission of a light pulse by the light source 20, and the shutter timing of the image sensor 30. The above-mentioned time difference will be herein sometimes referred to as “phase difference.” The “timing of emission” by the light source 20 refers to the timing of the start of rising of a light pulse emitted from the light source 20. The “shutter timing” refers to the timing to start exposure. The control circuit 60 may adjust the phase difference by varying the timing of emission, or may control the phase difference by varying the shutter timing.

The control circuit 60 may be, for example, a combination of a processor and a memory, or an integrated circuit such as a microcontroller incorporating a processor and a memory. The control circuit 60 adjusts, for example, the timing of emission and the shutter timing through execution, by the processor, of a program stored in the memory.

The signal processing circuit 70 processes a detection signal output from the image sensor 30. The signal processing circuit 70 performs computational processing such as image processing. The signal processing circuit 70 may be implemented by, for example, a digital signal processor (DSP), a programmable logic device (PLD) such as a field programmable gate array (FPGA), or a combination of a central processing unit (CPU) or graphics processing unit (GPU) and a computer program.

The control circuit 60 and the signal processing circuit 70 may be a single unitary circuit, or may be separate discrete circuits. The signal processing circuit 70 may be, for example, a component of an external apparatus, such as a server disposed at a remote location. In this case, the external apparatus such as a server transmits and receives data to and from the light source 20, the image sensor 30, and the control circuit 60 via wireless or wired communications.

The signal processing circuit 70 according to the embodiment is capable of, based on a detection signal output from the image sensor 30, generating moving image data representing the time variation of the respective concentrations of oxyhemoglobin (Oxy-Hb), deoxyhemoglobin (Deoxy-Hb), and total hemoglobin (Total-Hb) in blood in the interior of a target portion. The signal processing circuit 70 does not necessarily generate such moving image data but may generate other information. For example, the signal processing circuit 70 may be synchronized with another piece of equipment to generate biological information such as muscle oxygen consumption or blood oxygen saturation.

The signal processing circuit 70 may be designed to estimate an offset component due to disturbance light, and remove the offset component. The offset component refers to a signal component due to disturbance light, such as sunlight or fluorescent light. The offset component due to ambient light or disturbance light is estimated through detection of a signal by the image sensor 30 with the light source 20 being turned off, that is, with no light being emitted from the light source 20.

### 1-5. Other Components

The measurement apparatus 100 may include imaging optics for forming a two-dimensional image of the user on the light-receiving surface of the image sensor 30. The optical axis of the imaging optics is substantially orthogonal to the light-receiving surface of the image sensor 30. The imaging optics may include a zoom lens. A change in the location of the zoom lens causes a change in the magnification of the two-dimensional image of the user, which in turn causes a change in the resolution of the two-dimensional image on the image sensor 30. This ensures that even if the user is at a far distance, a desired measurement region can be magnified for detailed observation.

The measurement apparatus 100 may include a bandpass filter between the user and the image sensor 30. The bandpass filter is designed to pass only light within a wavelength range emitted by the light source 20, or light with wavelengths in the vicinity of the wavelength range. This helps to reduce the influence of ambient light or other disturbance components. The bandpass filter may be, for example, a multilayer filter or an absorption filter. In consideration of a band shift due to temperature variation of the light source 20 or oblique incidence on the filter, the bandpass filter may be designed to have a bandwidth ranging from, for example, about 20 nm to about 100 nm.

The measurement apparatus 100 may include a polarizer disposed between the light source 20 and the user, and a polarizer disposed between the image sensor 30 and the user. In this case, the polarization direction of the polarizer disposed near the light source 20, and the polarization direction of the polarizer disposed near the image sensor 30 may have a crossed-Nicols relationship. Such positioning helps to ensure that the regular reflection component contained in the surface reflection component associated with the user, that is, a component with the same angle of reflection as the angle of incidence, does not reach the image sensor 30. In other words, the quantity of the surface reflection component of light that reaches the image sensor 30 can be reduced.

### 2. Signal Detection Operation

Detailed reference will be made below to a signal detection operation performed by the measurement apparatus 100.

The measurement apparatus 100 according to the embodiment is capable of distinguishing between and detecting the surface reflection component and the internal scattering component that are contained in a reflected light pulse reflected off a target portion. If the target portion is an arm or leg, the signal intensity due to the internal scattering component to be detected is very small. As previously mentioned, this is due to radiation of a very small quantity of light that meets the laser safety standard, and to the large scattering and absorption by the skin and the subcutaneous fat. Further, the magnitude of a change in signal intensity associated with a change in blood flow or in blood components during muscular activity is very small, equivalent to several tenths of that of the above-mentioned signal intensity. This means that in detecting the internal scattering component, the surface reflection component, which is several thousand times to several ten thousand times greater in magnitude than the internal scattering component to be detected, is to be removed as much as possible.

Reference will be made below to an exemplary operation performed by the measurement apparatus 100 to detect the internal scattering component.

As described above, as the light source 20 irradiates a target portion of the user with a light pulse, the surface reflection component and the internal scattering component are generated. Part of each of the surface reflection component and the internal scattering component reaches the image sensor 30. The internal scattering component passes through the interior of the target portion before reaching the image sensor 30 after its emission from the light source 20. The optical path length of the internal scattering component is thus greater than the optical path length of the surface reflection component. Therefore, the timing of arrival of the internal scattering component at the image sensor 30 is on average later than the timing of arrival of the surface reflection component at the image sensor 30.

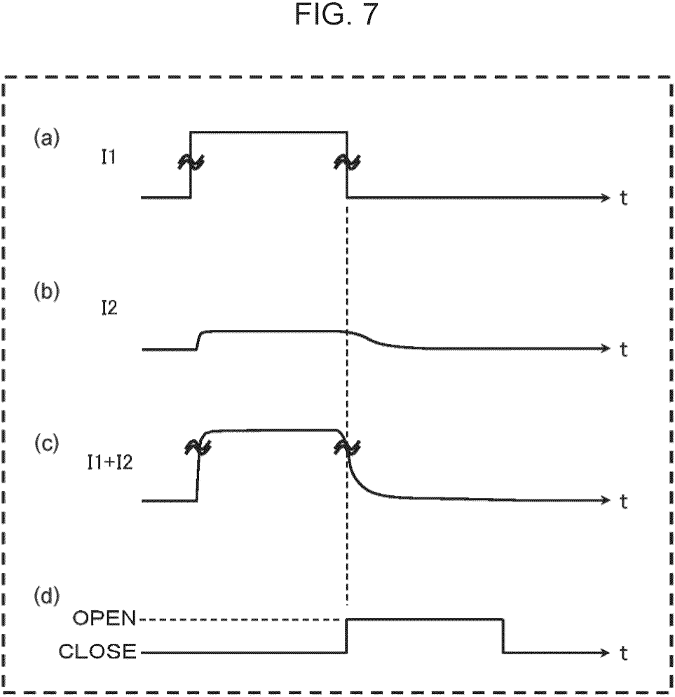

FIG. 7 schematically illustrates, for a case where a rectangular light pulse is emitted from the light source 20, waveforms of the optical intensity of a reflected light pulse reflected back from a target portion of the user. The horizontal axis in each of FIG. 7(a) to FIG. 7(d) represents time (t). The vertical axis represents intensity in each of FIG. 7(a) to FIG. 7(c), and represents the OPEN or CLOSE state of the electronic shutter in FIG. 7(d). FIG. 7(a) illustrates a surface reflection component I1. FIG. 7(b) illustrates an internal scattering component I2. FIG. 7(c) illustrates the sum component of the surface reflection component I1 and the internal scattering component I2. As illustrated in FIG. 7(a), the surface reflection component I1 maintains a substantially rectangular waveform. The internal scattering component I2, by contrast, is a combination of light rays with various optical path lengths. For this reason, as illustrated in FIG. 7(b), the internal scattering component I2 of the light pulse characteristically has a tail-like portion at the pulse trailing edge. In other words, the falling period of the internal scattering component I2 is longer than the falling period of the surface reflection component I1. To extract an increased proportion of the internal scattering component I2 from the light signal illustrated in FIG. 7(c), exposure with the electronic shutter is started at or after the arrival of the trailing edge of the surface reflection component I1 as illustrated in FIG. 7(d). In other words, exposure is started at or after the fall of the waveform of the surface reflection component I1. The shutter timing is controlled by the control circuit 60.

If a target portion is not flat, light arrives at a different time at each individual pixel of the image sensor 30. In this case, the shutter timing illustrated in FIG. 7(d) may be determined individually for each pixel. For example, a direction perpendicular to the light-receiving surface of the image sensor 30 is defined as z-direction. The control circuit 60 may acquire data representing the two-dimensional distribution of the z-coordinates of the surface of the target portion, and based on the acquired data, vary the shutter timing for each individual pixel. As a result, even if the target portion has a curved surface, the optimum shutter timing can be determined for each individual location on the surface. The data representing the two-dimensional distribution of the z-coordinates of the surface of the target portion is acquired by, for example, the time-of-flight (TOF) technique. This involves measuring the time required for arrival, at each individual pixel, of reflected light generated when light radiated by the light source 20 is reflected off the target portion. Based on the difference between the phase of the reflected light detected at each individual pixel, and the phase of the radiated light at the light source 20, the distance between the pixel and the target portion can be estimated. This makes it possible to acquire data representing the two-dimensional distribution of the z-coordinates of the surface of the target portion. The data representing the two-dimensional distribution may be acquired in advance prior to the measurement.

In the example illustrated in FIG. 7(a), the trailing edge of the surface reflection component I1 falls vertically. In other words, the time it takes for the fall of the surface reflection component I1 to end after its start is zero. In reality, however, there are cases where the trailing edge of the surface reflection component I1 does not fall vertically. For example, the trailing edge of the surface reflection component I1 does not fall vertically in one of the following cases; the falling of the waveform of a light pulse emitted from the light source 20 is not completely vertical; the target portion has minute irregularities on its surface; and scattering occurs in the epidermis. Since the user is a non-transparent object, the quantity of the surface reflection component I1 of light is very large relative to the quantity of the internal scattering component I2 of light. This means that if the trailing edge of the surface reflection component I1 extends even slightly beyond the time point of the vertical fall, this may cause the internal scattering component I2 to be buried. Further, there are also cases where a time delay associated with electron movement may occur during the readout period of the electronic shutter. For these reasons, an ideal binary readout like that illustrated in FIG. 7(d) may not be achievable in some cases. In such cases, the control circuit 60 may cause the shutter timing of the electronic shutter to be slightly later than a point immediately after the falling of the surface reflection component I1. For example, the control circuit 60 may cause the shutter timing of the electronic shutter to be about 0.5 ns to 5 ns later than the time point at which the falling of the surface reflection component I1 occurs. Instead of adjusting the shutter timing of the electronic shutter, the control circuit 60 may adjust the timing of emission by the light source 20. In other words, the control circuit 60 may adjust the time difference between the shutter timing of the electronic shutter and the timing of emission by the light source 20. For non-contact measurement of changes of blood flow or blood components within a target portion, if the shutter timing is delayed too much, the quantity of the internal scattering component I2, which is comparatively small in the first place, becomes even smaller. For this reason, the shutter timing may be allowed to remain in the vicinity of the trailing edge of the surface reflection component I1. As previously mentioned, a time delay due to internal scattering in the target portion is about 4 ns. In this case, the shutter timing may be delayed at maximum by about 4 ns.

As illustrated in the examples illustrated in FIGS. 6A and 6B, signals may be stored by performing exposures at shutter timings with the same time difference by using individual light pulses emitted from the light source 20. This allows for amplification of the quantity of the internal scattering component I2 of light.

Instead of or in addition to disposing a bandpass filter between the user and the image sensor 30, an offset component may be estimated by performing image capture with the same exposure time with no light being emitted by the light source 20. The estimated offset component is removed by being subtracted from a signal detected by each individual pixel of the image sensor 30. This allows for removal of a dark current component generated on the image sensor 30.

The internal scattering component I2 contains information on the internal characteristics of the user, for example, muscle oxygen consumption information. The quantity of light absorbed by the blood varies with temporal fluctuations in the user’s muscle oxygen consumption. This results in a corresponding increase or decrease in the quantity of light detected by the image sensor 30. Accordingly, monitoring the internal scattering component I2 makes it possible to estimate the time variation of the respective concentrations of oxyhemoglobin (Oxy-Hb), deoxyhemoglobin (Deoxy-Hb), and total hemoglobin (Total-Hb) in blood in the target portion of the user. The time variation of Oxy-Hb, Deoxy-Hb, and Total-Hb allows for estimation of muscle oxygen consumption.

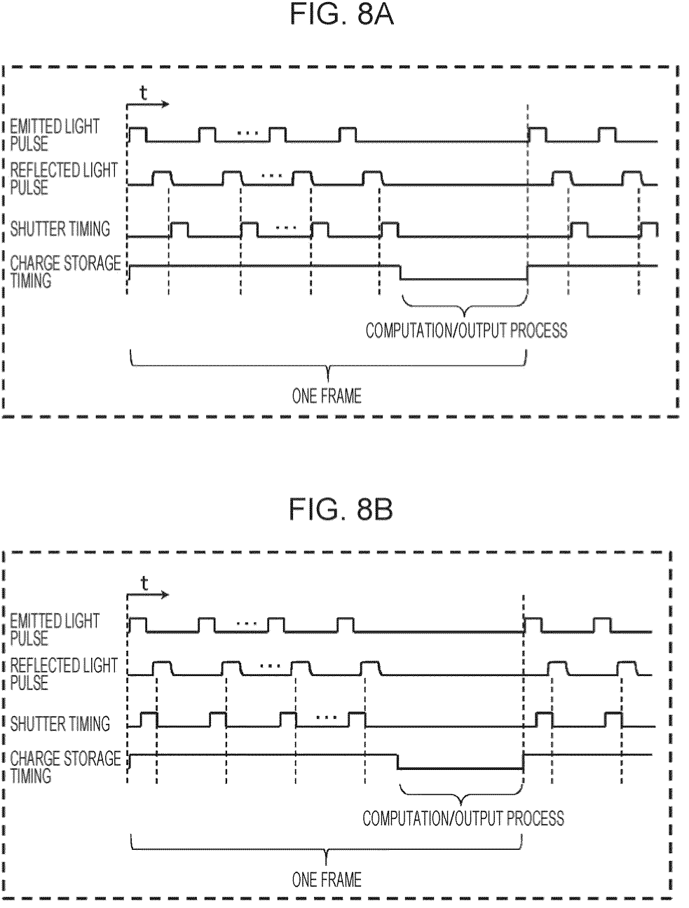

FIG. 8A is a timing chart illustrating an exemplary operation of detecting the internal scattering component I2. In the present example, the light source 20 repeatedly emits a light pulse within the period of one frame. The shutter of the image sensor 30 is set to the OPEN state in the period when the trailing edge of each reflected light pulse arrives at the image sensor 30. This operation allows the image sensor 30 to store a signal representative of the internal scattering component I2. Once signal storage has been performed a predetermined number of times, the image sensor 30 outputs a detection signal representing the signal stored for each individual pixel. The detection signal thus output is processed by the signal processing circuit 70.

As described above, the control circuit 60 repeats a detection operation including: causing the light source 20 to emit a light pulse; and causing the image sensor 30 to detect at least part of a component of a reflected light pulse after the start of falling of the reflected light pulse, and to output a detection signal representing the spatial distribution of the intensity of the internal scattering component. The above-mentioned operation allows the signal processing circuit 70 to, based on repeatedly output detection signals, generate and output distribution data representing the spatial distribution of muscle oxygen consumption in the target portion.

Reference will now be made to an exemplary method for detecting the surface reflection component I1. The surface reflection component I1 contains information on the surface characteristics of the user, for example, information on the skin blood flow in the arm or leg. Information on the surface reflection component I1 is not necessarily required but may be acquired as needed.

FIG. 8B is a timing chart illustrating an exemplary operation of detecting the surface reflection component I1. To detect the surface reflection component I1, the shutter of the image sensor 30 is set to the OPEN state before each of reflected light pulses arrives at the image sensor 30, and the shutter is set to the CLOSE state before the trailing edge of the reflected light pulse arrives at the image sensor 30. Controlling the shutter in this way helps to reduce the risk of the internal scattering component I2 mixing in the surface reflection component I1, and consequently increase the proportion of the surface reflection component I1. The CLOSE timing for the shutter may be set to a point immediately after the arrival of light at the image sensor 30. This allows for signal detection with an increased proportion of the surface reflection component I1, which has a relatively short optical path length. Acquiring a signal representative of the surface reflection component I1 allows for estimation of the pulse rate of the user, or the oxygenation of the epidermal blood flow of the user. Other methods for acquiring the surface reflection component I1 may be used, such as causing the image sensor 30 to detect the entirety of the reflected light pulse, or causing the image sensor 30 to detect continuous-wave light emitted from the light source 20.

The surface reflection component I1 may be detected by an apparatus other than the measurement apparatus 100 used to acquire the internal scattering component I2. For example, another device such as a sphygmograph or Doppler blood flow meter may be used. In that case, the other device is used with the following features taken into account: inter-device timing synchronization, optical interference, and calibration of detection sites. Performing time-division imaging by use of the same measurement apparatus 100 or the same sensor as in the embodiment may lead to reduced spatial and temporal inconsistencies. To acquire both a signal representing the surface reflection component I1 and a signal representing the internal scattering component I2 with the same sensor, the components to be acquired may be switched frame by frame as illustrated in FIGS. 8A and 8B. Alternatively, the components to be acquired may be alternately switched at high speed within one frame. This helps to reduce the time difference between the detection of the surface reflection component I1 and the detection of the internal scattering component I2.

Further, the respective signals of the surface reflection component I1 and the internal scattering component I2 may be acquired by use of two wavelengths of light. For example, two wavelengths of pulsed light, 750 nm and 850 nm, may be used. This allows changes in the respective concentrations of oxyhemoglobin and deoxyhemoglobin to be calculated from changes in the quantity of detected light at each of these wavelengths. An exemplary method that may be used to acquire the surface reflection component I1 and the internal scattering component I2 by use of two wavelengths is to switch between four types of charge storage within one frame as illustrated in FIG. 4 to 6A. Such a method helps to reduce temporal inconsistencies between the detection signals.

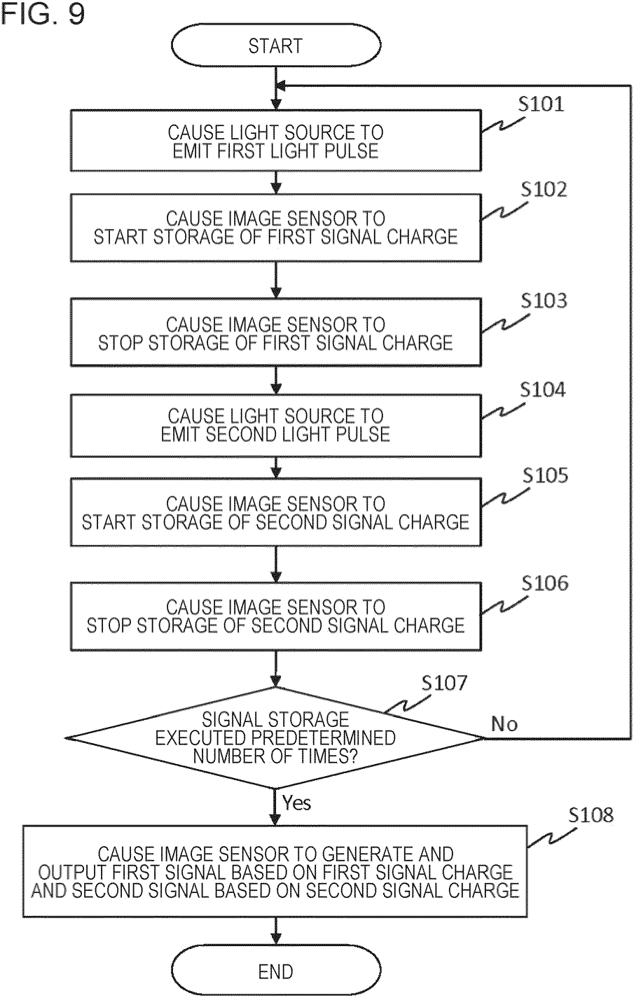

FIG. 9 is a flowchart illustrating an exemplary operation performed by the control circuit 60 to control the light source 20 and the image sensor 30. The following description is directed to an exemplary operation in which the light source 20 emits two wavelengths of pulsed light, and the image sensor 30 detects only the internal scattering component. The light source 20 emits a first light pulse with a first wavelength, and a second light pulse with a second wavelength. The first wavelength is greater than or equal to 650 nm and less than 805 nm. The second wavelength is greater than or equal to 805 nm and less than or equal to 950 nm.

At step S101, the control circuit 60 causes the light source 20 to emit the first light pulse for a predetermined time. At this time, the image sensor 30 is in a state in which exposure by the electronic shutter is stopped. The control circuit 60 causes the electronic shutter to stop exposure until the completion of a period during which the surface reflection component of a first reflected light pulse, which arises due to radiation of the first light pulse, arrives at the image sensor 30. Next, at step S102, the control circuit 60 causes the electronic shutter to start exposure at the timing when the internal scattering component of the first reflected light pulse arrives at the image sensor 30. After elapse of a predetermined time, at step S103, the control circuit 60 causes the electronic shutter to stop the exposure. Through steps S102 and S103, signal charge is stored in one of the first to fourth floating diffusions 204 to 207 illustrated in FIG. 5. The signal charge stored at this time will be hereinafter referred to as “first signal charge.”

At step S104, the control circuit 60 causes the light source 20 to emit the second light pulse for a predetermined time. At this time, the image sensor 30 is in a state in which exposure by the electronic shutter is stopped. The control circuit 60 causes the electronic shutter to stop exposure until the completion of a period during which the surface reflection component of a second reflected light pulse, which arises due to radiation of the second light pulse, arrives at the image sensor 30. Next, at step S105, the control circuit 60 causes the electronic shutter to start exposure at the timing of arrival, at the image sensor 30, of the internal scattering component of the second reflected light pulse. After elapse of a predetermined time, at step S106, the control circuit 60 causes the electronic shutter to stop the exposure. Through steps S 105 and S 106, a signal charge is stored in another one of the first to fourth floating diffusions 204 to 207 illustrated in FIG. 5. The signal charge stored at this time will be hereinafter referred to as “second signal charge.”

Subsequently, at step S107, the control circuit 60 determines whether the above-mentioned signal storage has been executed a predetermined number of times. If the result of the determination at step S107 is No, steps S101 to S106 are repeated until the result of the determination becomes Yes.

If the result of determination at step S107 is Yes, the process proceeds to step S108. At step S108, the control circuit 60 causes the image sensor 30 to generate and output a first signal based on the first signal charge, and a second signal based on the second signal charge.

As described above, the control circuit 60 executes a first operation of causing the light source 20 to emit the first light pulse, and causing the image sensor 30 to detect at least part of a component of the first reflected light pulse in the falling period of the first reflected light pulse. The control circuit 60 executes a second operation of causing the light source to emit the second light pulse, and causing the image sensor 30 to detect at least part of a component of the second reflected light pulse in the falling period of the second reflected light pulse. The control circuit 60 repeats a series of operations including the first operation and the second operation a predetermined number of times. Alternatively, the control circuit 60 may repeat the first operation a predetermined number of times, and then repeat the second operation a predetermined number of times. The first operation and the second operation may be interchanged in their order.

The operations illustrated in FIG. 9 allow the internal scattering component to be detected with high sensitivity. In irradiating a living body such as a human being with light to acquire information such as blood flow, the light undergoes a very large attenuation within the living body. For example, the light exiting the living body may, in some cases, be attenuated in intensity to about one millionth of the light entering the living body. Thus, in some cases, radiation of a single pulse alone may not provide a sufficient quantity of light for detecting the internal scattering component. For radiation according to Class 1 of the laser safety standard, in particular, the quantity of light provided is very small. Accordingly, in the example illustrated in FIG. 9, the light source 20 emits a light pulse multiple times, and the image sensor 30 is likewise exposed multiple times by means of the electronic shutter. Through such operations, the detection signal can be integrated for improved sensitivity. Executing emission of light and exposure multiple times is not necessarily required but may be performed as needed.

Although FIG. 9 illustrates an example in which the internal scattering component is detected, in another example, the surface reflection component may be further detected. If both the surface reflection component and the internal scattering component are to be detected, a step of storing a signal charge based on the surface reflection component of the first reflected light pulse is added between step S101 and step S102, and a step of storing a signal charge based on the surface reflection component of the second reflected light pulse is added between step S104 and step S105. These signal charges are each stored in the corresponding one of the two remaining floating diffusions of the first to fourth floating diffusions 204 to 207 illustrated in FIG. 5. Detecting the surface reflection component makes it possible to, for example, acquire information representing the user’s appearance or the state of the user’s skin blood flow.

Reference will now be made to an exemplary operation performed by the signal processing circuit 70 to estimate muscle oxygen consumption.

As previously mentioned, oxyhemoglobin and deoxyhemoglobin differ in their light absorption characteristics. Oxyhemoglobin has relatively high absorption of infrared light with wavelengths above approximately 805 nm. Deoxyhemoglobin, by contrast, has relatively high absorption of near-infrared or red light with wavelengths below 805 nm. For near-infrared light with wavelengths near 805 nm, oxyhemoglobin and deoxyhemoglobin have substantially the same absorptivity. Accordingly, the first wavelength of greater than or equal to 650 nm and less than 805 nm, and the second wavelength of greater than or equal to 805 nm and less than or equal to 950 nm may be used. For example, the two wavelengths of light mentioned above, 750 nm and 850 nm, may be used. Based on how much of each wavelength of light is detected, the time variation of the respective concentrations of oxyhemoglobin and deoxyhemoglobin can be detected.

The signal processing circuit 70 generates and outputs a signal representing the state of blood inside a target portion, based on a first detection signal and a second detection signal that are output from the image sensor 30. The first detection signal is a signal representing the detection results on the internal scattering component of the first wavelength (e.g., 750 nm) of light. The second detection signal is a signal representing the detection results on the internal scattering component of the second wavelength (e.g., 850 nm) of light. By solving a predetermined system of equations by use of the first detection signal and the second detection signal, the signal processing circuit 70 is able to determine how much the respective concentrations of oxyhemoglobin (HbO₂) and deoxyhemoglobin (Hb) in blood have changed from their initial values. The system of equations is given by, for example, Equations (1) and (2) below.

$${ε}_{O X Y}^{750} Δ H b {O}_{2} + {ε}_{d e O X Y}^{750} Δ H b = - l n \frac{{\text{I}}_{n o w}^{750}}{{\text{I}}_{i n i}^{750}}$$

$${ε}_{O X Y}^{850} Δ H b {O}_{2} + {ε}_{d e O X Y}^{850} Δ H b = - l n \frac{{\text{I}}_{n o w}^{850}}{{\text{I}}_{i n i}^{850}}$$

ΔHbO₂ and ΔHb respectively represent how much the concentrations of HbO₂ and Hb in blood have changed from their initial values. ε⁷⁵⁰_{OXY} and ε⁷⁵⁰_{deOXY} respectively represent the molar absorption coefficients of HbO₂ and Hb at the wavelength of 750 nm. ε⁸⁵⁰_{OXY} and ε⁸⁵⁰_{deOXY} respectively represent the molar absorption coefficients of HbO₂ and Hb at the wavelength of 850 nm. I⁷⁵⁰ini and I⁷⁵⁰_{now} respectively represent the detected intensities at the initial instant and at the instant of measurement with respect to the wavelength of 750 nm. I⁸⁵⁰ini and I⁸⁵⁰_{nOw} respectively represent the detected intensities at the initial instant and at the instant of measurement with respect to the wavelength of 850 nm. For example, the signal processing circuit 70 is capable of, based on Equations (1) and (2) above, calculating, for each individual pixel, changes ΔHbO₂ and ΔHb in the respective concentrations of HbO₂ and Hb in blood from the initial values. This allows for generation of data representing the two-dimensional distribution of changes in the respective concentrations of HbO₂ and Hb in blood in the target portion.

The signal processing circuit 70 is further capable of determining the oxygen saturation of hemoglobin. Oxygen saturation is a value representing how much proportion of hemoglobin in blood is bound to oxygen. Oxygen saturation is defined by the equation below, where C(Deoxy-Hb) represents the concentration of deoxyhemoglobin, and C(Oxy-Hb) represents the concentration of oxyhemoglobin.

Oxygen saturation = C(Oxy-Hb) / [C(Oxy-Hb) + C(Deoxy-Hb)] x 100(%)

A living body contains components other than blood that absorb red light and near-infrared light. Nevertheless, temporal fluctuations in light absorptivity are mainly due to hemoglobin in the arterial blood. Therefore, blood oxygen saturation can be measured with high accuracy based on fluctuations in absorptivity.

Light passes through the skin and the subcutaneous tissue before reaching the muscle. This means that the detection results also contain, as superimposed information, blood flow fluctuations in the skin and the subcutaneous tissue. To remove or reduce the influence of such superimposed information, the signal processing circuit 70 may perform a process of subtracting the surface reflection component I1 from the internal scattering component 12 detected by the image sensor 30. This allows for acquisition of pure muscle blood flow information excluding the skin and subcutaneous-tissue blood flow information. The subtraction in this case may be performed by, for example, a method of subtracting a value from the signal of the internal scattering component I2, the value being a multiplication of the signal of the surface reflection component I1 by a given coefficient of greater than or equal to one, which is determined by taking the difference in optical path length into account. The coefficient may be, for example, found by a simulation or an experiment based on the average optical constants of typical human arms or legs. Such a subtraction process can be easily executed for cases where measurement is to be performed with the same measurement apparatus 100 or the same sensor and by use of the same wavelength of light. This is because of the ability to easily reduce temporal and spatial inconsistencies, and the ability to easily match up the skin and subcutaneous-tissue blood flow components contained in the internal scattering component 12 with the characteristics of the surface reflection component 11.

The two-dimensional distribution of skin and subcutaneous-tissue blood flow is independent of the two-dimensional distribution of muscle blood flow. Accordingly, based on signals output from the image sensor 30, the two-dimensional distribution of the internal scattering component I2, and the two-dimensional distribution of the surface reflection component I1 may be separated from each other by use of a statistical technique such as independent component analysis or principal component analysis.

### 3. Exemplary Detection of Changes in Muscle Blood Flow

Reference will now be made to an exemplary method for detecting changes in the muscle blood flow of the user.

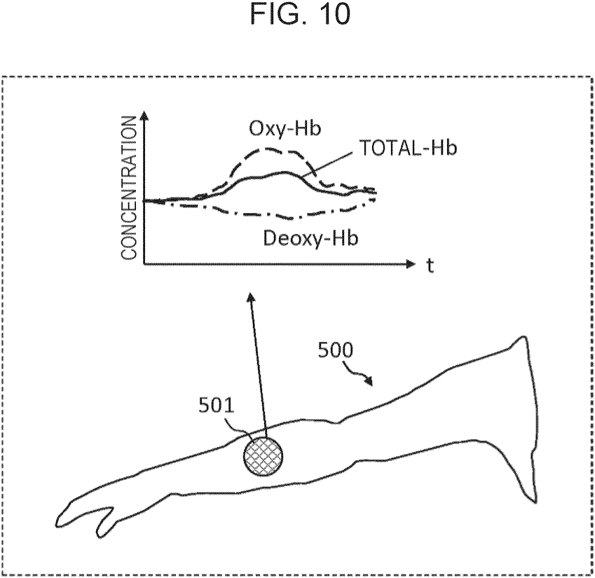

FIG. 10 schematically illustrates an example of the time variation of muscle blood flow. As illustrated in FIG. 10, a target portion 501 of a user 500 is irradiated with the light from the light source 20, and the light returning from the target portion 501 is detected. In this case, the surface reflection component is very large in magnitude relative to the internal scattering component. Fortunately, however, only the internal scattering component can be extracted through the shutter adjustment previously mentioned. The graph in FIG. 10 represents an example of the time course of the respective concentrations of oxyhemoglobin (Oxy-Hb) and deoxyhemoglobin (Deoxy-Hb) in blood inside the target portion 501, and an example of the time course of the sum (Total-Hb) of these concentrations. The internal scattering component in this example is acquired by use of two wavelengths of light. The concentrations in FIG. 10 represent changes from the normal baseline values. Such changes are calculated by the signal processing circuit 70 based on light intensity signals.

Blood flow in a muscle reflects balance between oxygen consumption and oxygen supply in the muscle’s cells. Thus, in comparison to the resting condition, in the early stages of exercise, oxygen consumption exceeds oxygen supply, and oxygen concentration consequently decreases. During exercise, oxygen concentration becomes constant if the balance between oxygen supply and oxygen consumption is maintained. After exercise, oxygen supply exceeds oxygen consumption, and oxygen concentration consequently increases. Thus, according to the embodiment, the time course of muscle blood flow is measured at the same location within the target portion 501 of the user 500. In observation of the time course of muscular activity, even if the absolute value of muscle blood flow is unknown, the state of the user’s muscular activity can be estimated from temporal relative changes in muscle blood flow.

FIG. 11 schematically illustrates an exemplary case where measurements are made simultaneously at multiple locations within the target portion 501 of the user 500. In the present example, light is radiated to a region that extends two-dimensionally, and light scattered inside the two-dimensional region is detected. This makes it possible to acquire data representing the two-dimensional distribution of the blood oxygenation state inside the target portion 501. In this case, the pattern of radiation from the light source 20 may exhibit, for example, a uniform distribution with even intensity, a dotted distribution, or a doughnut-shaped distribution. If the radiation from the light source 20 has a uniform distribution with even intensity, this makes it possible to obviate or simplify the adjustment of the location of irradiation on the target portion 501. Radiation with a uniform distribution allows light to be incident on the target portion 501 of the user 500 from a broad area. This helps to enhance the signal detected by the image sensor 30. This further allows for measurement at any location within a region to be irradiated.

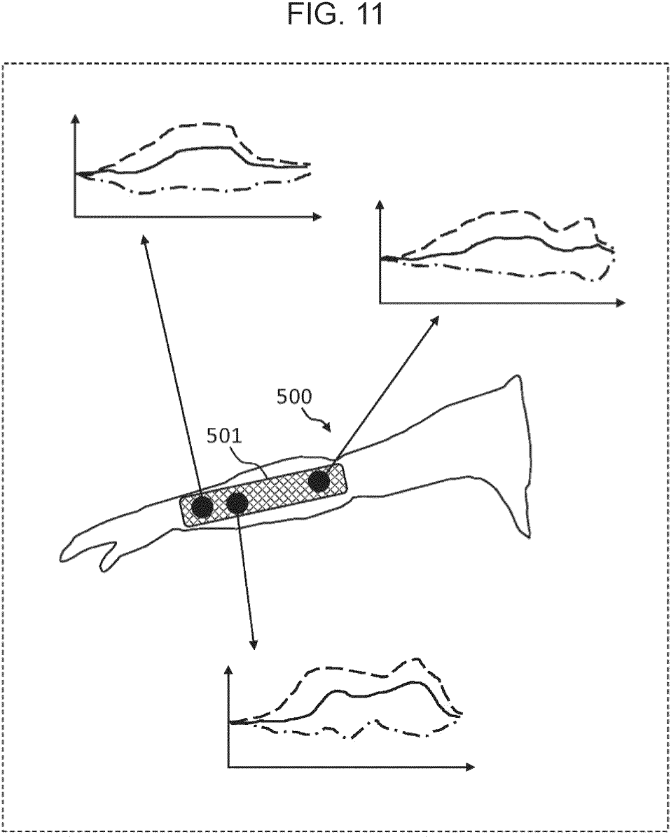

FIG. 12 schematically illustrates an exemplary irradiation region 22 to be irradiated with light. In non-contact measurement of a living body, the quantity of detected light attenuates in inverse proportion to the square of the distance from the measurement apparatus 100 to the target portion. Accordingly, the signal of each individual pixel generated by the image sensor 30 may be enhanced by integrating the signals of neighboring pixels. This helps to reduce the number of integrated pulses while maintaining the SN ratio. This allows for improved frame rate.

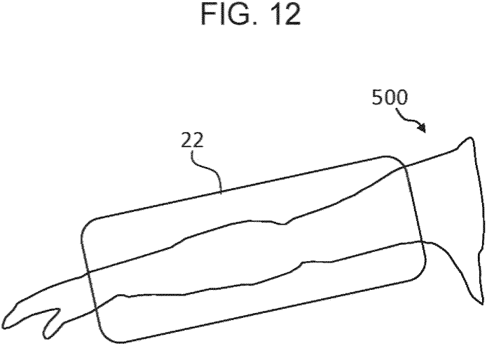

FIG. 13 schematically illustrates how a signal changes in response to a lateral shift in the location of a target portion of the user 500. As previously mentioned, a reading of a change of muscular activity is provided by detecting how much the concentration of oxyhemoglobin or deoxyhemoglobin in blood changes in response to a change in the state of muscular activity from the resting condition. Using the image sensor 30 with a two-dimensional array of photoelectric converters makes it possible to acquire information on the two-dimensional distribution of muscular activity. According to the embodiment, measurement is made in a non-contact manner. Thus, as illustrated in the lower illustration in FIG. 13, the location of the target portion may change during measurement in some cases. This may occur, for example, even with a slight movement of the user 500 due to breathing. Generally, the two-dimensional distribution of oxyhemoglobin and deoxyhemoglobin concentrations in muscle blood does not change abruptly within an infinitesimal time. Consequently, for example, displacement of the target portion can be corrected through inter-frame pattern matching of the detected two-dimensional distribution. Alternatively, for periodic movements such as breathing, only the corresponding frequency component may be extracted for correction or removal. The target portion is not necessarily a single region but may be made up of multiple regions. For example, such multiple regions may be two regions, one on the left and one on the right, or may have a dotted distribution in two by six matrix form.

It is also possible to estimate muscular activity from changes in blood state in two periods. Specifically, measurement is made before training, and after training is done, measurement is made again. Then, the state of blood in muscle is compared and analyzed before and after training to allow determination or evaluation of the effectiveness of training. In that case, changes in level of oxyhemoglobin and other components in muscle blood at the same point are compared before and after training. If such a measurement method is to employed, the signal processing circuit 70 is capable of extracting one or more features from an appearance image representing the appearance of the user including the target portion, detecting displacement of the target portion from the relative position between the one or more features and a measurement region or an analysis region, and correcting the measurement region to thereby reduce or compensate for the influence of such displacement. Such a displacement correction may be performed during the measurement, or may be performed during an analysis performed after the measurement is finished.

An image including the target portion of the user may be, for example, an infrared image generated based on a light pulse emitted from the light source 20 and reflected back from the target portion. The image may be generated based on a component including at least part of the leading edge component or trailing edge component of a light pulse that has arrived at the image sensor 30. Alternatively, the image of the target portion may be a visible image acquired by a camera or sensor that is disposed separately from the image sensor 30 of the measurement apparatus 100.

Non-limiting examples of the above-mentioned features may include moles, the outline of the target portion, extremities such as the fingertips, nails, and blood vessel shapes. A marker serving as a feature may be made on the target portion. The signal processing circuit 70 is capable of, through pattern matching using one or more such features, detecting a change in the location of the target portion before and after exercise. The signal processing circuit 70 is capable of, after performing a process that compensates for the detected change in location, generating distribution data representing the spatial distribution of muscle oxygen consumption.

### 4. Determination of Training Effectiveness

Reference will now be made to a specific method for determining the effectiveness of training for the user 500 by use of the measurement apparatus 100.

As previously mentioned, based on the detection results on the internal scattering component of reflected light, the state of blood in the vicinity of the muscle tissue of the user 500 can be estimated. By exploiting this characteristic, muscle oxygen consumption of the user 500 can be calculated. Based on the results of the muscle oxygen consumption measurement, the effectiveness of muscle exercise such as strength training or rehabilitation can be determined.

According to the embodiment, cuff compression by the compression unit 40 to occlude blood flow is used as an exemplary method for measuring muscle oxygen consumption of the user 500. The light source 20 repeatedly emits a light pulse toward the arm or leg of the user 500 at predetermined intervals of time. The image sensor 30 stores, for each individual pixel, a signal representing the internal scattering component of the reflection of the light pulse. According to the embodiment, the same operation as mentioned above is performed in each of the following periods: during rest before the start of cuff compression; during cuff compression; and during rest after release of cuff compression.

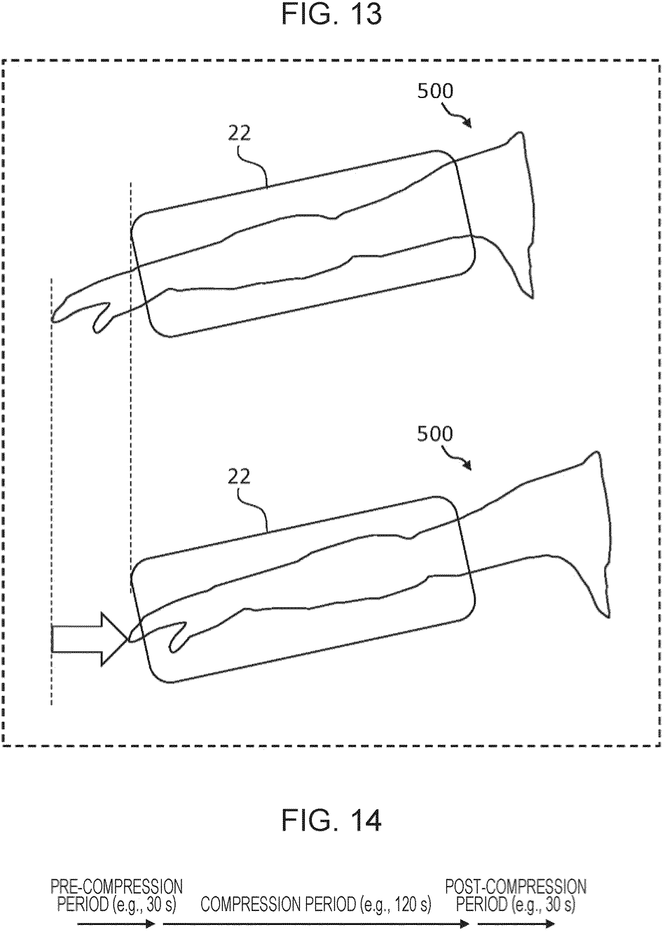

FIG. 14 illustrates how a single measurement operation of muscle oxygen consumption proceeds along the time axis. A single measurement operation according to the embodiment includes the following three periods: a pre-compression period, a compression period, and a post-compression period. The pre-compression period refers to the period before the start of cuff compression. The compression period refers to the period during cuff compression. The post-compression period refers to the period immediately after the release of cuff compression.

Over these three periods, the measurement apparatus 100 repeatedly generates a detection signal representative of the user’s muscular activity. During the compression period, the user uses a cuff to compress a predetermined site such as the arm or the leg. The compression pressure to be applied may be set to, for example, a predetermined value such as 40 mmHg or 200 mmHg. In the example in FIG. 14, the pre-compression period and the post-compression period each has a duration of 30 seconds, and the compression period has a duration of 120 seconds. The respective durations of these periods are not limited to those exemplified above but may be adjusted as appropriate. The pre-compression period and the post-compression period may have different durations.

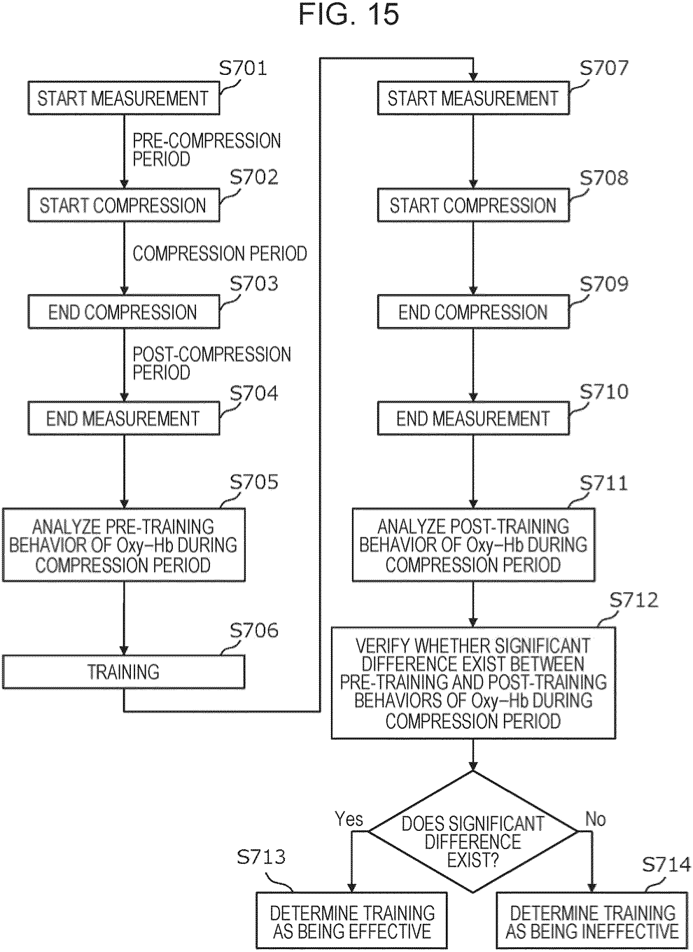

FIG. 15 is a flowchart illustrating an exemplary operation performed by the measurement apparatus 100 to determine training effectiveness. The control circuit 60 according to the embodiment repeatedly executes the signal detection operation mentioned above in each of a first period and a second period. The first period is the period before the user performs muscle exercise. The second period is the period after the user performs muscle exercise. The signal processing circuit 70 generates distribution data, which represents the spatial distribution of muscle oxygen consumption, based on detection signals repeatedly output from the image sensor 30 in the first period and on detection signals repeatedly output from the image sensor 30 in the second period. More specifically, the signal processing circuit 70 generates, based on detection signals repeatedly output in the first period, first blood flow data representing the time course of the concentration distribution of oxyhemoglobin in blood inside the target portion. The signal processing circuit 70 generates, based on detection signals repeatedly output in the second period, second blood flow data representing the time course of the concentration distribution of oxyhemoglobin in blood inside the target portion. Based on the first blood flow data and the second blood flow data, the signal processing circuit 70 generates distribution data representing the two-dimensional distribution of muscle oxygen consumption. For example, the signal processing circuit 70 generates a first rate of change from the first blood flow data. The first rate of change represents the slope of decrease in the time variation of the concentration of oxyhemoglobin at multiple points included in the target portion. The signal processing circuit 70 generates a second rate of change from the second blood flow data. The second rate of change represents the slope of decrease in the time variation of the concentration of oxyhemoglobin at multiple points included in the target portion. Based on the difference or ratio between the first rate of change and the second rate of change, the muscle oxygen consumption at each individual point on the target portion can be estimated.

In the example in FIG. 15, when the user performs an operation for starting measurement, the measurement apparatus 100 starts a measurement operation (step S701). At this point, cuff compression of a part of the user’s body with the compression unit 40 is not performed. With the user doing nothing, a detection signal is acquired repeatedly over a predetermined time (e.g., 30 seconds). This is performed to acquire data representing the user’s resting blood flow. This period corresponds to the pre-compression period mentioned above. As previously mentioned, the measurement is made by the light source 20 emitting a light pulse toward the user’s target portion such as the arm or leg, and by the image sensor 30 detecting the leading edge component of the light pulse reflected by the target portion. The internal scattering component of the reflected light pulse is thus detected repeatedly. The image sensor 30 outputs, for each individual pixel, a detection signal corresponding to the amount of the reflected light pulse stored. The signal processing circuit 70 repeatedly generates, based on the detection signal output from the image sensor 30, blood flow data representing the concentration distribution of oxyhemoglobin distribution. The blood flow data may be generated, for example, for individual pixels or for individual groups of pixels.

Upon elapse of a predetermined time from the start of measurement, the control circuit 60 causes the compression unit 40 to start compression of a part of the user’s body (step S702). The blood flow data continues to be generated while the compression is applied. While the compression is applied to the user, the signal processing circuit 70 repeatedly generates blood flow data based on the detection signal output from the image sensor 30.

Upon elapse of a predetermined time (e.g., 120 seconds) from the start of the compression applied with the cuff (to be referred to as “cuff compression” hereinafter), the cuff compression ends, and the part of the user’s body is released from the cuff compression (step S703). The control circuit 60 causes the compression unit 10 to stop the compression. Even after the compression is stopped, the measurement is continued over a predetermined time (e.g., 30 seconds). Blood flow data is thus repeatedly generated even in the period after the end of cuff compression.

Upon elapse of a predetermined time from the end of cuff compression, the control circuit 60 ends the measurement (step S704). Subsequently, the signal processing circuit 70 analyzes blood flow data acquired in each of the pre-compression period, the compression period, and the post-compression period. The signal processing circuit 70 thus analyzes the time variation of Oxy-Hb concentration in the compression period (step S705). For example, with the analysis start point defined as the maximum point of Oxy-Hb reached following an increase immediately after the start of cuff compression, the signal processing circuit 70 calculates the slope, that is, the rate of change, of decrease in Oxy-Hb. Specifically, the signal processing circuit 70 executes a process described below, including:
(1) identifying the maximum point of Oxy-Hb during the compression period;
(2) with the maximum point as the analysis start point, performing linear fitting for the behavior of Oxy-Hb over a predetermined period from the analysis start point, the predetermined period being set to, for example, a value such as 30 seconds or 60 seconds; and
(3) determining the slope of the linear fitted line.

Through the process mentioned above, the signal processing circuit 70 calculates the slope of decrease in Oxy-Hb during the compression period. The slope of decrease in Oxy-Hb during the compression period represents the amount of muscle oxygen consumption. A small slope of decrease indicates small muscle oxygen consumption, and a large slope of decrease indicates large muscle oxygen consumption. This completes the measurement operation for muscle oxygen consumption before training.

Subsequently, the user performs training such as strength training or rehabilitation (step S706). Then, muscle oxygen consumption after training is measured in the same manner as mentioned above (steps S707 to S711). Steps S707 to S711 are respectively similar to steps S701 to S705. At step S711, the signal processing circuit 70 calculates the slope of decrease in Oxy-Hb during the compression period after training.

Thus, a comparison can be made between pre-training muscle oxygen consumption and post-training muscle oxygen consumption. From the pre-training muscle oxygen consumption and the post-training muscle oxygen consumption, the signal processing circuit 70 verifies, by use of an existing verification method, whether a significant difference exists between these two values (step S712). If a significant difference exists, the signal processing circuit 70 determines that the training performed is effective (S713). Conversely, if no significant difference exists, the signal processing circuit 70 determines that the training performed is ineffective (step S714). For example, if the result of comparison between the pre-training muscle oxygen consumption and the post-training muscle oxygen consumption reveals that the amount of increase in muscle oxygen consumption due to the training exceeds a reference value, the signal processing circuit 70 may determine that the training is effective, and if the result of comparison reveals that the amount of increase in muscle oxygen consumption due to the training does not exceed the reference value, the signal processing circuit 70 may determine that the training is ineffective. The determination of whether the training is effective may be performed for each individual pixel. The signal processing circuit 70 may, for each individual pixel, determine the amount of increase in muscle oxygen consumption that serves as the basis for the above-mentioned determination result, and generate image data represented in a color that varies according to the level of the increase. For example, the signal processing circuit 70 may generate an image such that those pixels for which an increase in muscle oxygen consumption exceeds a reference value are represented in red to indicate high training effectiveness, and those pixels for which an increase in muscle oxygen consumption does not exceed the reference value is represented in blue to indicate low training effectiveness. Alternatively, the signal processing circuit 70 may generate an image that is color coded in three or more colors according to the level of the increase in muscle oxygen consumption. Such an image may be displayed on the display 50 or the AR glass 90. A color bar indicating the correspondence between each color and training effectiveness may be displayed.

The above-mentioned process allows the user to, based on whether there has been an increase in muscle oxygen consumption, visually recognize the effectiveness of training in the form of a two-dimensional distribution. The signal processing circuit 70 may generate an image that is a superimposition of two images, one being an appearance image representing the user’s appearance acquired with the image sensor 30 or another device, and one being an image representing the two-dimensional distribution of increases in muscle oxygen consumption. Display of such an image allows the user to clearly recognize the effectiveness of training at each individual muscle site. An image representing the two-dimensional distribution of increases in muscle oxygen consumption may be displayed so as to overlap a real image of the user that is visible through the AR glass 90. Such display allows the user to more clearly recognize the effectiveness of training at each individual muscle site.

The signal processing circuit 70 may determine whether there has been an increase in muscle oxygen consumption, based on the magnitude of the slope of decrease in Oxy-Hb before and after exercise. For example, the signal processing circuit 70 may, if the slope of Oxy-Hb after exercise (i.e., the second rate of change) is greater than or equal to a factor “a” (which is a real number greater than one) of the slope of Oxy-Hb before exercise (i.e., the first rate of change), determine that the muscle oxygen consumption is relatively large or that the training performed is highly effective, and add information to that effect to the distribution data of muscle oxygen consumption and output the resulting data. The factor “a” may be set to, for example, a suitable value such as two. The signal processing circuit 70 may generate an image such that a region with a large or small increase in muscle oxygen consumption is highlighted. Display of such an image allows the user to recognize the effectiveness of training with improved accuracy.

According to the embodiment, from the two-dimensional distribution of increases in muscle oxygen consumption, the user is able to recognize those muscle sites for which training is effective and those muscle sites for which training is ineffective, and provide feedback for the planning of the next training menu. This allows for more effective training. The signal processing circuit 70 may, from the two-dimensional distribution of increases in muscle oxygen consumption, generate a training plan for training a region for which an increase in muscle oxygen consumption is determined to be relatively small, and display the generated training plan on the display 50 or the AR glass 90. In that case, data is pre-stored in the storage medium 80 in the form of, for example, a table that defines the correspondence between muscle sites and one or more training items for stimulating each of the muscle sites. The signal processing circuit 70 is capable of, by referencing the stored data, generating a training plan for strengthening those muscle sites for which an increase in muscle oxygen consumption is relatively small. This allows the user to improve the effectiveness of training by executing the generated training plan presented to the user. According to the embodiment, even users without sufficient knowledge about training are able to execute training with improved effectiveness.

The signal processing circuit 70 may, in providing a training plan, reference information on the user’s training history, check the training history against training effectiveness, and create and provide a more optimized training plan. Further, the signal processing circuit 70 may, in providing a training plan, reference user identification information, and provide an effective training plan that is more suited to each individual user. For such a configuration, user identification information, and training history information for each individual user are pre-stored in the storage medium 80.

### Example

Reference will be made below to an example of a method for evaluating the effectiveness of training based on the measurements of muscle oxygen consumption.

FIG. 16 illustrates an experiment for evaluating the effectiveness of training by use of a NIRS camera 400. In this experiment, the blood flow dynamics during blood flow occlusion before and after training are measured with the NIRS camera 400. The NIRS camera 400 corresponds to the measurement apparatus 100 according to the embodiment mentioned above. The measurement is made at the forearm muscle, and the training involves performing 100 repetitions of handgrip exercise with a hand gripper 450 (load: 2 kg). Blood flow occlusion is performed by compressing the upper arm with the cuff at a pressure of 200 mmHg.

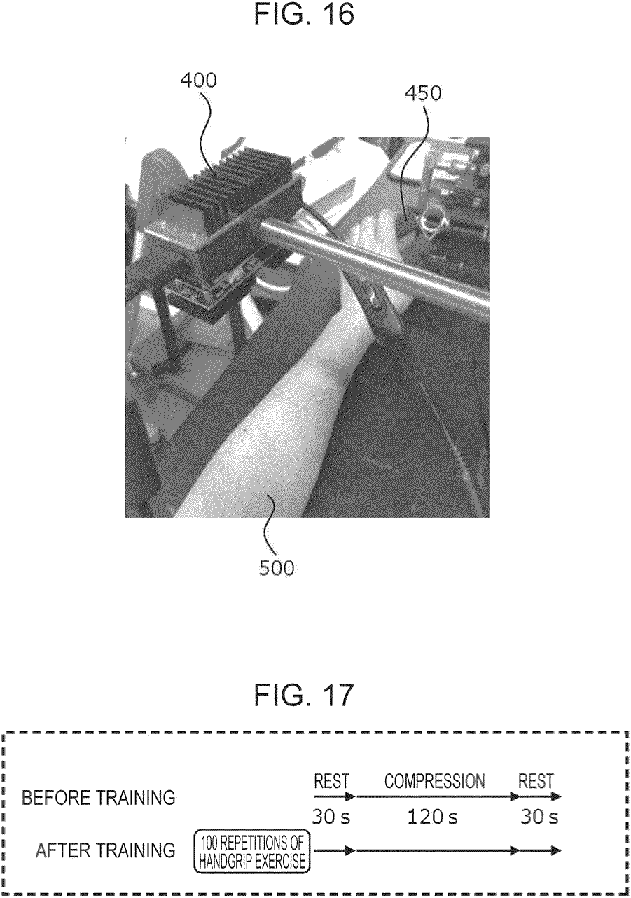

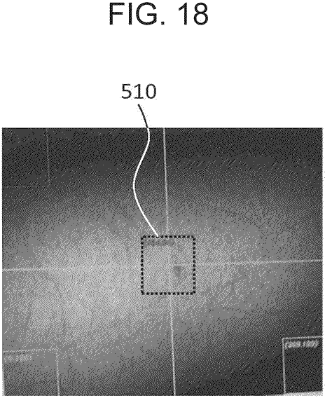

FIG. 17 illustrates an experiment protocol. The experiment protocol begins with 30 seconds of rest, followed by 120 seconds of cuff compression, which is then followed by 30 seconds of rest. The blood flow dynamics are measured before training with the NIRS camera 400, and then training is performed with the hand gripper 450. Immediately after the training, the blood flow dynamics are measured again under the same protocol as mentioned above. To ensure consistency of the measurement location before and after the training, a marking is made on the forearm with a permanent marker. FIG. 18 illustrates an infrared image captured with the NIRS camera 400. Alignment is performed based on a marked location 510 in the infrared image illustrated in FIG. 18. In this way, accurate alignment can be performed based on the location of a feature within the image captured by the NIRS camera 400. This is one of the major advantages of the measurement method according to the embodiment of the present disclosure over existing methods that involve placing the NIRS apparatus directly on the skin.

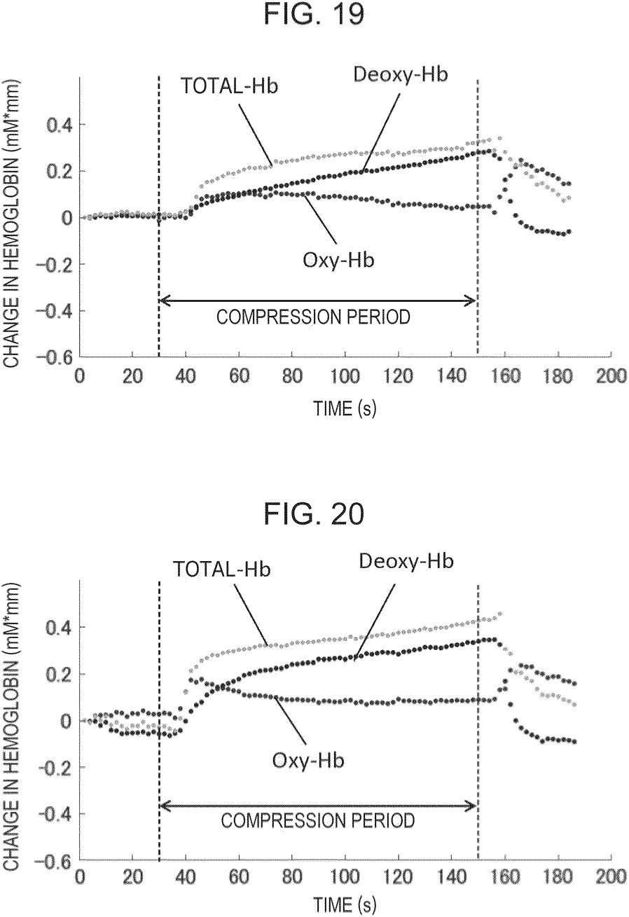

FIGS. 19 and 20 illustrate the time variation of changes in hemoglobin level measured with the NIRS camera 400 before and after training, respectively. FIGS. 19 and 20 each illustrate the time variation of changes in the respective concentrations of oxyhemoglobin (Oxy-Hb), deoxyhemoglobin (Deoxy-Hb), and total hemoglobin (Total-Hb) from the corresponding baseline values. Both before and after training, Total-Hb increases significantly in response to the start of compression, increases gradually while the compression is applied, and decreases after release of the compression. Oxy-Hb increases significantly in response to the start of compression, decreases gradually while the compression is applied, increases significantly immediately after release of the compression, and then decreases. Deoxy-Hb increases slightly at the start of compression, increases gradually while the compression is applied, increases significantly immediately after release of the compression, and then decreases significantly after release of the compression. As described above, Oxy-Hb and Deoxy-Hb are observed to exhibit different behaviors in each of the above-mentioned periods, that is, at the start of compression, during compression, and at the release of compression. With focus now directed to the period during the application of compression, in this period, Oxy-Hb decreases gradually, and Deoxy-Hb increases gradually. This is presumably due to consumption, by the metabolism of the muscle cells, of oxygen in the blood that has pooled in the forearm as a result of blood flow occlusion caused by cuff compression of the upper arm. As for the general blood flow dynamics mentioned above, similar tendencies are observed before and after the training.

The experiment is conducted under the hypothesis that “an increase in muscle oxygen consumption due to training leads to an increase in the slope of decrease in Oxy-Hb during cuff compression.” From a comparison between the pre-training and post-training slopes of decrease in Oxy-Hb respectively illustrated in FIGS. 19 and 20, it can be confirmed that the magnitude of the slope of decrease in Oxy-Hb during the compression period measured after the training does increase relative to that of the pre-training slope.

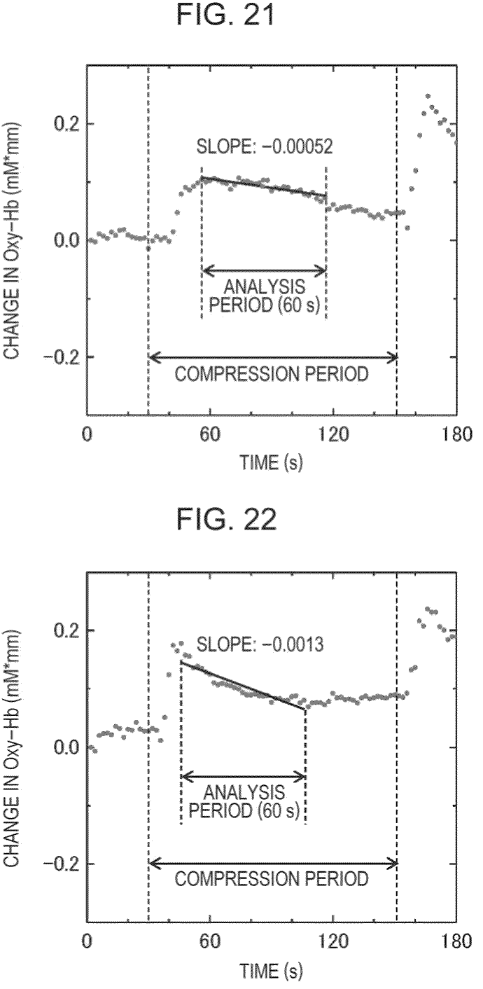

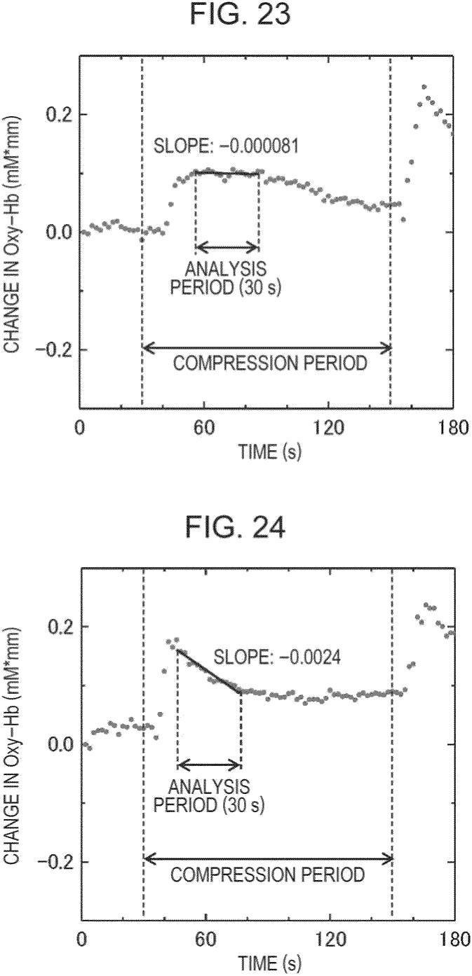

For quantitative discussion of the slope of decrease in Oxy-Hb during cuff compression before and after training, a detailed analysis is made on the behaviors of Oxy-Hb during cuff compression illustrated in FIGS. 19 and 20. FIGS. 21 to 24 illustrate the results of analysis on the behaviors of Oxy-Hb during cuff compression. FIGS. 21 and 22 respectively illustrate the results of analysis on the pre-training and post-training behaviors of Oxy-Hb for a case where the analysis period is 60 seconds. FIGS. 23 and 24 respectively illustrate the results of analysis on the pre-training and post-training behaviors of Oxy-Hb for a case where the analysis period is 30 seconds. In each of these examples, the analysis start point is defined as the time point of the maximum change in Oxy-Hb. The analysis end point is 60 seconds after the analysis start point in the examples in FIGS. 21 and 22, and is 30 seconds after the analysis start point in the examples in FIGS. 23 and 24. As illustrated in FIGS. 21 to 24, the cuff-compression start point and the analysis start point are different from each other. Since there is a time lag between the time point of the start of cuff compression and the time point of the maximum change in Oxy-Hb, setting the cuff-compression start point as the analysis start point does not allow for accurate calculation of the slope of decrease in Oxy-Hb.

As illustrated in FIGS. 21 to 24, it has been confirmed that since the decrease in Oxy-Hb during compression is not linear, the fitting accuracy is comparatively low for the case where the analysis period is 60 seconds, whereas comparatively good fitting is achieved for the case where the analysis period is 30 seconds. It has been thus confirmed that, as described above, by setting the analysis start point as the time point of the maximum change in Oxy-Hb, and setting the analysis period to 30 seconds, the slope of decrease in Oxy-Hb during cuff compression can be analyzed with improved accuracy.

As illustrated in FIGS. 21 and 22, for the case where the analysis period is 60 seconds, the slope of decrease in Oxy-Hb during compression before training is -0.00052, whereas the corresponding slope after training is -0.0013, which indicates an increase in absolute value by a factor of about 2.5. A comparison between the plot and the fitted line illustrated in each of FIGS. 21 and 22 reveals that the deviation between the plot and the fitted line is particularly large after training. This indicates that the fitted line does not correctly represent the actual slope in the early stages of Oxy-Hb decrease. For the case where the analysis period is 30 seconds, by contrast, as illustrated in FIGS. 23 and 24, the slope of decrease in Oxy-Hb during compression before training is -0.000081, whereas the corresponding slope after training is -0.0024, which indicates a significant increase by a factor of about 30.0. Further, for the case where the analysis period is 30 seconds, the plot and the fitted line agree well with respect to the slope in the early stages of Oxy-Hb decrease. The above results confirm that setting a shorter analysis period such as 30 seconds allows for more accurate calculation of the slope of decrease in Oxy-Hb during compression.

Although the example uses linear fitting, fitting with a function different from a straight line may be used. The signal processing circuit may, in each measurement period before and after training, fit the time variation of oxyhemoglobin concentration in a predetermined period following the end of increase in oxyhemoglobin concentration to a predetermined function, and determine the rate of change from the time rate of change of the function.

### Other Embodiments

According to the embodiment and the example described above, muscle oxygen consumption is measured with blood flow in the target portion being occluded by use of the compression unit 40. However, the present disclosure is not necessarily limited to such a method. Reference will be made below to another exemplary method for measuring muscle oxygen consumption.

A method used for muscle oxygen consumption estimation other than the blood flow occlusion method is to estimate muscle oxygen consumption based on the difference between Oxy-Hb and Deoxy-Hb after training. During training, muscle oxygen consumption increases in comparison to the pre-training level, which causes the oxygen consumption to increase relative to the oxygen supply in the blood. This results in decreased Oxy-Hb relative to the pre-training level, and increased Deoxy-Hb relative to the pre-training level. After training, to compensate for the shortage of oxygen in the blood in the muscle, Oxy-Hb increases, and Deoxy-Hb decreases. Now, the difference between the value of Oxy-Hb and the value of Deoxy-Hb after training is defined as ΔHb (= Oxy-Hb -Deoxy-Hb). The value of ΔHb may be used as an index representing a measure of the effectiveness of training. That is, the value of ΔHb increases with increasing muscular load of the training performed. Therefore, based on the two-dimensional distribution of AHb, sites that have been effectively trained and sites that have not been effectively trained can be visualized two-dimensionally.

A still another method is to use, as an index of training effectiveness, the ratio of Oxy-Hb to Total-Hb (= Oxy-Hb + Deoxy-Hb) after training. During training, muscle oxygen consumption increases, which causes the oxygen consumption to increase relative to the oxygen supply in the blood. This results in a decreased ratio of Oxy-Hb to Total-Hb during training relative to the pre-training level. After training, to compensate for the shortage of oxygen in the blood in the muscle, Oxy-Hb increases, which causes the ratio of Oxy-Hb to Total-Hb to increase. Now, the time it takes for the ratio of Oxy-Hb to Total-Hb to return to the baseline, pre-training level at this time is defined as recovery time. The recovery time provides information about muscle oxygen consumption. Specifically, the recovery time represents aerobic capacity, that is, exercise capacity. Increased aerobic capacity due to training performed on a continuous basis leads to reduced recovery time. Accordingly, continuously measuring recovery time with respect to a muscle site of interest makes it possible to, after elapse of a predetermined period of time, two-dimensionally visualize at which muscle site the aerobic capacity has or has not improved.

The technique according to the present disclosure enables non-contact acquisition of information on the user’s muscular activity. The technique according to the present disclosure finds utility in a variety of apparatuses, including cameras, measurement instruments, smartphones, tablet computers, and head-mounted apparatuses.

What is claimed is:

1. A measurement apparatus for measuring muscle oxygen consumption in a target portion of a user who performs a muscle exercise, the measurement apparatus comprising:
a light source;
a sensor including photoelectric converters; and
a processing circuit that
executes a detection operation multiple times to acquire multiple detection signals, the detection operation including
causing the light source to emit a light pulse, and
causing the sensor to detect at least part of an internal scattering component of a reflected light pulse, and to output a detection signal representing a spatial distribution of intensity of the at least part of the internal scattering component, the reflected light pulse arising from the target portion due to emission of the light pulse, the internal scattering component being a component scattered in an interior of the target portion, the detection signal being included in the multiple detection signals, and
based on the multiple detection signals, generates and outputs distribution data, the distribution data representing a spatial distribution of muscle oxygen consumption in the target portion.

2. The measurement apparatus according to claim 1,
wherein the processing circuit generates, as the distribution data, image data that represents the spatial distribution of the muscle oxygen consumption in the target portion in a color that varies according to a level of the muscle oxygen consumption.

3. The measurement apparatus according to claim 1,
wherein the processing circuit generates, as the distribution data, image data representing an image, the image including an appearance image and information superimposed on the appearance image, the appearance image being acquired by the sensor or another device and representing an appearance of the user including the target portion, the information representing the spatial distribution of the muscle oxygen consumption.

4. The measurement apparatus according to claim 1,
wherein the processing circuit,
based on the multiple detection signals, estimates a concentration distribution of oxyhemoglobin in blood in the interior of the target portion, and
based on time variation of the concentration distribution of the oxyhemoglobin, estimates the spatial distribution of the muscle oxygen consumption.

5. The measurement apparatus according to claim 4,
wherein the processing circuit, based on a slope of time variation of concentration of the oxyhemoglobin, estimates the muscle oxygen consumption.

6. The measurement apparatus according to claim 1, further comprising
a compression unit,
wherein the processing circuit executes the detection operation with blood flow in the target portion being restricted through compression of a part of a body of the user by the compression unit.

7. The measurement apparatus according to claim 6,
wherein the compression unit is controlled by the processing circuit, and
wherein the processing circuit,
before executing the detection operation, causes the compression unit to start the compression of the part of the body of the user, and
after executing the detection operation, causes the compression unit to end the compression.

8. The measurement apparatus according to claim 1,
wherein the processing circuit
executes the detection operation multiple times in each of a first period and a second period, the first period being a period before the user performs the muscle exercise, the second period being a period after the user performs the muscle exercise, and
based on the multiple detection signals acquired in the first period, and the multiple detection signals acquired in the second period, generates the distribution data.

9. The measurement apparatus according to claim 8,
wherein the processing circuit
acquires data of an appearance image representing an appearance of the user including the target portion, the appearance image being acquired in each of the first period and the second period by the sensor or another device,
detects a change in location of the target portion between the first period and the second period, the change in location being detected by matching between one or more features included in the appearance image acquired in the first period, and the one or more features included in the appearance image acquired in the second period, and
after applying a process to the multiple detection signals acquired in the first period and to the multiple detection signals acquired in the second period, generates the distribution data, the process compensating for the change in location.

10. The measurement apparatus according to claim 8,
wherein the processing circuit,
based on the multiple detection signals acquired in the first period, generates first blood flow data, the first blood flow data representing time course of concentration distribution of oxyhemoglobin in blood in the interior of the target portion,
based on the multiple detection signals acquired in the second period, generates second blood flow data, the second blood flow data representing time course of concentration distribution of oxyhemoglobin in blood in the interior of the target portion, and
based on the first blood flow data and the second blood flow data, generates the distribution data.

11. The measurement apparatus according to claim 10,
wherein the processing circuit,
based on the first blood flow data, determines a first rate of change, the first rate of change representing a slope of decrease in time variation of concentration of the oxyhemoglobin at multiple points included in the target portion,
based on the second blood flow data, determines a second rate of change, the second rate of change representing a slope of decrease in time variation of concentration of the oxyhemoglobin at the multiple points, and
based on a difference or ratio between the first rate of change and the second rate of change, estimates the muscle oxygen consumption at the multiple points.

12. The measurement apparatus according to claim 11,
wherein the detection operation in each of the first period and the second period is executed with blood flow in the target portion being restricted through compression of a part of a body of the user, and
wherein the processing circuit,
in the first period, fits time variation of concentration of the oxyhemoglobin in a predetermined period to a function, and determines the first rate of change from a time rate of change of the function, the predetermined period being a predetermined period after an increase in concentration of the oxyhemoglobin ends, and
in the second period, fits time variation of concentration of the oxyhemoglobin in the predetermined period to the function, and determines the second rate of change from a time rate of change of the function.

13. The measurement apparatus according to claim 11,
wherein the processing circuit,
in response to the second rate of change being greater than or equal to a factor “a” of the first rate of change, adds, to the distribution data to be output, information indicating that the muscle oxygen consumption is relatively large, the factor “a” being a real number greater than one, and
in response to the second rate of change being less than the factor “a” of the first rate of change, adds, to the distribution data to be output, information indicating that the muscle oxygen consumption is relatively small.

14. The measurement apparatus according to claim 13,
wherein the processing circuit,
based on the information indicating that the muscle oxygen consumption is relatively large, determines a first region in the target portion, the first region being a region where the muscle oxygen consumption is relatively large, and
based on the information indicating that the muscle oxygen consumption is relatively small, determines a second region in the target portion, the second region being a region where the muscle oxygen consumption is relatively small, and
wherein the distribution data includes an image that highlights the first region or the second region.

15. The measurement apparatus according to claim 1,
wherein the processing circuit further generates and outputs data representing a training plan used to train a muscle in a region where the muscle oxygen consumption is relatively small, the region being included in the target portion.

16. The measurement apparatus according to claim 15,
wherein the processing circuit
acquires history data representing information on the muscle exercise performed by the user, and
based on the history data, adjusts the training plan.

17. The measurement apparatus according to claim 15,
wherein the processing circuit
acquires identification data that identifies the user, and
based on the identification data, adjusts the training plan.

18. The measurement apparatus according to claim 1,
wherein the light source emits a first light pulse and a second light pulse, the first light pulse having a first wavelength of greater than or equal to 650 nm and less than 805 nm, the second light pulse having a second wavelength of greater than or equal to 805 nm and less than or equal to 950 nm,
wherein the detection operation includes
causing the light source to emit the first light pulse,
causing the sensor to detect at least part of a first internal scattering component of a first reflected light pulse, and to output a first detection signal representing a spatial distribution of intensity of the at least part of the first internal scattering component, the first reflected light pulse arising from the target portion due to emission of the first light pulse, the first internal scattering component being a component scattered in the interior of the target portion,
causing the light source to emit the second light pulse, and
causing the sensor to detect at least part of a second internal scattering component of a second reflected light pulse, and to output a second detection signal representing a spatial distribution of intensity of the at least part of the second internal scattering component, the second reflected light pulse arising from the target portion due to emission of the second light pulse, the second internal scattering component being a component scattered in the interior of the target portion, and
wherein the processing circuit,
based on the first detection signal and the second detection signal, estimates a concentration distribution of oxyhemoglobin in blood in the interior of the target portion, and
based on time variation of the concentration distribution of the oxyhemoglobin, estimates the spatial distribution of the muscle oxygen consumption.

19. The measurement apparatus according to claim 1, further comprising
an augmented reality glass including a transparent display,
wherein the transparent display displays a distribution image representing the distribution data, the distribution image being superimposed on an appearance of the user that is visible through the transparent display.

20. The measurement apparatus according to claim 1,
wherein the internal scattering component is a component of the reflected light pulse that is detected after start of decrease in intensity of the reflected light pulse.

21. A method executable by a computer, the computer being included in a measurement apparatus that measures muscle oxygen consumption in a target portion of a user who performs a muscle exercise, the method comprising:
executing a detection operation multiple times to acquire multiple detection signals, the detection operation including
causing a light source to emit a light pulse, and
causing a sensor to detect at least part of an internal scattering component of a reflected light pulse, and to output a detection signal representing a spatial distribution of intensity of the at least part of the internal scattering component, the sensor including photoelectric converters, the reflected light pulse arising from the target portion due to emission of the light pulse, the internal scattering component being a component scattered in an interior of the target portion, the detection signal being included in the multiple detection signals; and
based on the multiple detection signals, generating and outputting distribution data, the distribution data representing a spatial distribution of muscle oxygen consumption in the target portion.

22. A non-transitory computer-readable recording medium storing a computer program executable by a computer, the computer being included in a measurement apparatus that measures muscle oxygen consumption in a target portion of a user who performs a muscle exercise, the computer program causing the computer to execute a process, the process comprising:
executing a detection operation multiple times to acquire multiple detection signals, the detection operation including
causing a light source to emit a light pulse, and
causing a sensor to detect at least part of an internal scattering component of a reflected light pulse, and to output a detection signal representing a spatial distribution of intensity of the at least part of the internal scattering component, the sensor including photoelectric converters, the reflected light pulse arising from the target portion due to emission of the light pulse, the internal scattering component being a component scattered in an interior of the target portion, the detection signal being included in the multiple detection signals; and
based on the multiple detection signals, generating and outputting distribution data, the distribution data representing a spatial distribution of muscle oxygen consumption in the target portion.
